SEQUENCE LISTING (97 sequences)

SEQ ID NO: 1 - protein, 393 aa, Homo sapiens [sequence omitted]

SEQ ID NO: 2 - protein, 5 aa, Homo sapiens
TTLSN

SEQ ID NO: 3 - protein, 7 aa, Homo sapiens
LVKSGEV

SEQ ID NO: 4 - protein, 14 aa, Homo sapiens
CAESYSGGYQKVTF

SEQ ID NO: 5 - protein, 6 aa, Homo sapiens
ISNHLY

SEQ ID NO: 6 - protein, 6 aa, Homo sapiens
FYNNEI

SEQ ID NO: 7 - protein, 12 aa, Homo sapiens
CASSSFSNEQFF

SEQ ID NO: 8 - protein, 111 aa, Homo sapiens
QQVMQIPQYQHVQEGEDFTTYCNSSTTLSNIQWYKQRPGGHPVFLIQLVKSGEVKKQKRLTFQFGEAKKNSSLHITATQTTDVGTYFCAESYSGGYQKVTFGIGTKLQVIP

SEQ ID NO: 9 - protein, 112 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSSFSNEQFFGPGTRLTVL

SEQ ID NO: 10 - protein, 130 aa, Artificial Sequence
  Synthetic
MHLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAESYSGGYQKVTFGIGTKLQVIP

SEQ ID NO: 11 - protein, 131 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSSFSNEQFFGPGTRLTVL

SEQ ID NO: 12 - protein, 267 aa, Artificial Sequence
  Synthetic
MHLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAESYSGGYQKVTFGIGTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 13 - protein, 304 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSSFSNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 14 - protein, 248 aa, Artificial Sequence
  Synthetic
QQVMQIPQYQHVQEGEDFTTYCNSSTTLSNIQWYKQRPGGHPVFLIQLVKSGEVKKQKRLTFQFGEAKKNSSLHITATQTTDVGTYFCAESYSGGYQKVTFGIGTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 15 - protein, 285 aa, Artificial Sequence
  Synthetic
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSSFSNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 16 - protein, 598 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSSFSNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMHLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAESYSGGYQKVTFGIGTKLQVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 17 - protein, 7 aa, Homo sapiens
TSENNYY

SEQ ID NO: 18 - protein, 8 aa, Homo sapiens
QEAYKQQN

SEQ ID NO: 19 - protein, 15 aa, Homo sapiens
CAFMAYMEYGNKLVF

SEQ ID NO: 20 - protein, 5 aa, Homo sapiens
SNHLY

SEQ ID NO: 21 - protein, 6 aa, Homo sapiens
FYNNEI

SEQ ID NO: 22 - protein, 12 aa, Homo sapiens
CACKGITDTQYF

SEQ ID NO: 23 - protein, 115 aa, Homo sapiens
QTVTQSQPEMSVQEAETVTLSCTYDTSENNYYLFWYKQPPSRQMILVIRQEAYKQQNATENRFSVNFQKAAKSFSLKISDSQLGDTAMYFCAFMAYMEYGNKLVFGAGTILRVKS

SEQ ID NO: 24 - protein, 112 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCACKGITDTQYFGPGTRLTVL

SEQ ID NO: 25 - protein, 136 aa, Artificial Sequence
  Synthetic
MHRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMAYMEYGNKLVFGAGTILRVKS

SEQ ID NO: 26 - protein, 131 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCACKGITDTQYFGPGTRLTVL

SEQ ID NO: 27 - protein, 273 aa, Artificial Sequence
  Synthetic
MHRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMAYMEYGNKLVFGAGTILRVKSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 28 - protein, 304 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCACKGITDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 29 - protein, 252 aa, Artificial Sequence
  Synthetic
QTVTQSQPEMSVQEAETVTLSCTYDTSENNYYLFWYKQPPSRQMILVIRQEAYKQQNATENRFSVNFQKAAKSFSLKISDSQLGDTAMYFCAFMAYMEYGNKLVFGAGTILRVKSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 30 - protein, 285 aa, Artificial Sequence
  Synthetic
EPEVTQTPSHQVTQMGQEVILRCVPISNHLYFYWYRQILGQKVEFLVSFYNNEISEKSEIFDDQFSVERPDGSNFTLKIRSTKLEDSAMYFCACKGITDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 31 - protein, 604 aa, Artificial Sequence
  Synthetic
MATWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCACKGITDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMHRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMAYMEYGNKLVFGAGTILRVKSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 32 - protein, 6 aa, Homo sapiens
YGGTVN

SEQ ID NO: 33 - protein, 8 aa, Homo sapiens
YFSGDPLV

SEQ ID NO: 34 - protein, 15 aa, Homo sapiens
CAVTFMDTGRRALTF

SEQ ID NO: 35 - protein, 5 aa, Homo sapiens
SGHTA

SEQ ID NO: 36 - protein, 6 aa, Homo sapiens
FQGNSA

SEQ ID NO: 37 - protein, 14 aa, Homo sapiens
CASSPRGGDYEQYF

SEQ ID NO: 38 - protein, 114 aa, Homo sapiens
QSVSQHNHHVILSEAASLELGCNYSYGGTVNLFWYVQYPGQHLQLLLKYFSGDPLVKGIKGFEAEFIKSKFSFNLRKPSVQWSDTAEYFCAVTFMDTGRRALTFGSGTRLQVQP

SEQ ID NO: 39 - protein, 112 aa, Homo sapiens
GVSQSPSNKVTEKGKDVELRCDPISGHTALYWYRQSLGQGLEFLIYFQGNSAPDKSGLPSDRFSAERTGGSVSTLTIQRTQQEDSAVYLCASSPRGGDYEQYFGPGTRLTVT

SEQ ID NO: 40 - protein, 134 aa, Artificial Sequence
  Synthetic
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVTFMDTGRRALTFGSGTRLQVQP

SEQ ID NO: 41 - protein, 133 aa, Artificial Sequence
  Synthetic
MATRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSPRGGDYEQYFGPGTRLTVT

SEQ ID NO: 42 - protein, 271 aa, Artificial Sequence
  Synthetic
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVTFMDTGRRALTFGSGTRLQVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 43 - protein, 306 aa, Artificial Sequence
  Synthetic
MATRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSPRGGDYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 44 - protein, 251 aa, Artificial Sequence
  Synthetic
QSVSQHNHHVILSEAASLELGCNYSYGGTVNLFWYVQYPGQHLQLLLKYFSGDPLVKGIKGFEAEFIKSKFSFNLRKPSVQWSDTAEYFCAVTFMDTGRRALTFGSGTRLQVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 45 - protein, 285 aa, Artificial Sequence
  Synthetic
GVSQSPSNKVTEKGKDVELRCDPISGHTALYWYRQSLGQGLEFLIYFQGNSAPDKSGLPSDRFSAERTGGSVSTLTIQRTQQEDSAVYLCASSPRGGDYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 46 - protein, 604 aa, Artificial Sequence
  Synthetic
MATRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSPRGGDYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVTFMDTGRRALTFGSGTRLQVQPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 47 - protein, 6 aa, Homo sapiens
DRGSQS

SEQ ID NO: 48 - protein, 6 aa, Homo sapiens
IYSNGD

SEQ ID NO: 49 - protein, 15 aa, Homo sapiens
CAVRGGNTGFQKLVF

SEQ ID NO: 50 - protein, 5 aa, Homo sapiens
SQVTM

SEQ ID NO: 51 - protein, 7 aa, Homo sapiens
ANQGSEA

SEQ ID NO: 52 - protein, 11 aa, Homo sapiens
CSVRSEDTQYF

SEQ ID NO: 53 - protein, 114 aa, Homo sapiens
QQKEVEQNSGPLSVPEGAIASLNCTYSDRGSQSFFWYRQYSGKSPELIMFIYSNGDKEDGRFTAQLNKASQYVSLLIRDSQPSDSATYLCAVRGGNTGFQKLVFGTGTRLLVSP

SEQ ID NO: 54 - protein, 111 aa, Homo sapiens
AVISQKPSRDICQRGTSLTIQCQVDSQVTMMFWYRQQPGQSLTLIATANQGSEATYESGFVIDKFPISRPNLTFSTLTVSNMSPEDSSIYLCSVRSEDTQYFGPGTRLTVL

SEQ ID NO: 55 - protein, 134 aa, Artificial Sequence
  Synthetic
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVRGGNTGFQKLVFGTGTRLLVSP

SEQ ID NO: 56 - protein, 127 aa, Artificial Sequence
  Synthetic
MASLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVRSEDTQYFGPGTRLTVL

SEQ ID NO: 57 - protein, 271 aa, Artificial Sequence
  Synthetic
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVRGGNTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 58 - protein, 300 aa, Artificial Sequence
  Synthetic
MASLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVRSEDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 59 - protein, 251 aa, Artificial Sequence
  Synthetic
QQKEVEQNSGPLSVPEGAIASLNCTYSDRGSQSFFWYRQYSGKSPELIMFIYSNGDKEDGRFTAQLNKASQYVSLLIRDSQPSDSATYLCAVRGGNTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 60 - protein, 284 aa, Artificial Sequence
  Synthetic
AVISQKPSRDICQRGTSLTIQCQVDSQVTMMFWYRQQPGQSLTLIATANQGSEATYESGFVIDKFPISRPNLTFSTLTVSNMSPEDSSIYLCSVRSEDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 61 - protein, 598 aa, Artificial Sequence
  Synthetic
MASLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVRSEDTQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVRGGNTGFQKLVFGTGTRLLVSPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 62 - protein, 6 aa, Homo sapiens
VSNAYN

SEQ ID NO: 63 - protein, 4 aa, Homo sapiens
GSKP

SEQ ID NO: 64 - protein, 16 aa, Homo sapiens
CAVSGYQLIWGAGTKL

SEQ ID NO: 65 - protein, 5 aa, Homo sapiens
SGHAT

SEQ ID NO: 66 - protein, 6 aa, Homo sapiens
FQNNGV

SEQ ID NO: 67 - protein, 16 aa, Homo sapiens
CASSLDRRGGRETQYF

SEQ ID NO: 68 - protein, 112 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAVSGYQLIWGAGTKLIIKPNIQNPEPAV

SEQ ID NO: 69 - protein, 114 aa, Homo sapiens
GVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRRGGRETQYFGPGTRLLVL

SEQ ID NO: 70 - protein, 137 aa, Artificial Sequence
  Synthetic
MHLQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVSGYQLIWGAGTKLIIKPNIQNPEPAV

SEQ ID NO: 71 - protein, 135 aa, Artificial Sequence
  Synthetic
MATRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRRGGRETQYFGPGTRLLVL

SEQ ID NO: 72 - protein, 274 aa, Artificial Sequence
  Synthetic
MHLQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVSGYQLIWGAGTKLIIKPNIQNPEPAVNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 73 - protein, 308 aa, Artificial Sequence
  Synthetic
MATRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRRGGRETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 74 - protein, 249 aa, Artificial Sequence
  Synthetic
KDQVFQPSTVASSEGAVVEIFCNHSVSNAYNFFWYLHFPGCAPRLLVKGSKPSQQGRYNMTYERFSSSLLILQVREADAAVYYCAVSGYQLIWGAGTKLIIKPNIQNPEPAVNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 75 - protein, 287 aa, Artificial Sequence
  Synthetic
GVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRRGGRETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 76 - protein, 609 aa, Artificial Sequence
  Synthetic
MATRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLDRRGGRETQYFGPGTRLLVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMHLQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVSGYQLIWGAGTKLIIKPNIQNPEPAVNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 77 - protein, 137 aa, Artificial Sequence
  Synthetic
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 78 - protein, 137 aa, Artificial Sequence
  Synthetic
DIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 79 - protein, 173 aa, Artificial Sequence
  Synthetic
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 80 - protein, 27 aa, Artificial Sequence
  Synthetic
RAKRSGSGATNFSLLKQAGDVEENPGP

SEQ ID NO: 81 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 82 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 83 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 84 - protein, 302 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 85 - protein, 307 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 86 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 87 - protein, 209 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 88 - protein, 214 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 89 - protein, 25 aa, Homo sapiens
TCTYSPALNKMFCQLAKTCPVQLWV

SEQ ID NO: 90 - protein, 25 aa, Homo sapiens
TCTYSPALNKMFYQLAKTCPVQLWV

SEQ ID NO: 91 - protein, 9 aa, Homo sapiens
HMTEVVRRC

SEQ ID NO: 92 - protein, 9 aa, Homo sapiens
HMTEVVRHC

SEQ ID NO: 93 - protein, 25 aa, Homo sapiens
VGSDCTTIHYNYMCNSSCMGGMNRR

SEQ ID NO: 94 - protein, 25 aa, Homo sapiens
VGSDCTTIHYNYICNSSCMGGMNRR

SEQ ID NO: 95 - protein, 4 aa, Artificial Sequence
  Synthetic
RAKR

SEQ ID NO: 96 - protein, 25 aa, Homo sapiens
NVLYSPALNKMFYQLAKTCPVQLWV

SEQ ID NO: 97 - protein, 25 aa, Homo sapiens
SGTAKSVTCTMFYQLAKTCPVQLWV

(19) United States
(12) Patent Application Publication
(10) Pub. No.: US 2024/0254190 A1
(43) Pub. Date: Aug. 1, 2024

(54) T CELL RECEPTORS RECOGNIZING C135Y, R175H, OR M237I MUTATION IN P53

(71) Applicant: The United States of America, as represented by the Secretary, Department of Health and Human, Bethesda, MD (US)

(72) Inventors: Sanghyun Kim, Rockville, MD (US); Nikolaos Zacharakis, Gaithersburg, MD (US); Steven A. Rosenberg, Potomac, MD (US); Frank J. Lowery, III, Clarksburg, MD (US); Maria R. Parkhurst, Ellicott City, MD (US)

(73) Assignee: The United States of America, as represented by the Secretary, Department of Health and Human, Bethesda, MD (US)

(21) Appl. No.: 18/289,596

(22) PCT Filed: May 6, 2022
(86) PCT No.: PCT/US2022/028066

§ 371 Date: Nov. 6, 2023

Related U.S. Application Data
(60) Provisional application No. 63/185,805, filed on May 7, 2021.

(51) Int. Cl. C07K 14/725; A61K 39/00; A61P 35/00; C12N 5/0783; G01N 33/574

(52) U.S. Cl. CPC C07K 14/7051; A61K 39/4611; A61K 39/4632; A61K 39/464451; A61P 35/00; C12N 5/0636; G01N 33/574; C12N 2510/00

(57) ABSTRACT

Disclosed are isolated or purified T cell receptors (TCRs) having antigenic specificity for human p53^{C135Y}, human p53^{R175H}, or human p53^{M237I}. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS-REFERENCE TO RELATED APPLICATION

This patent application claims the benefit of U.S. Provisional Patent Application No. 63/185,805, filed May 7, 2021, which is incorporated by reference in its entirety herein.

### STATEMENT REGARDING FEDERALLY SPONSORED RESEARCH OR DEVELOPMENT

This invention was made with Government support under project number BC010985 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 133,457 Byte ASCII (Text) file named “759875_ST25.txt,” dated Apr. 21, 2022.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, pancreatic, colorectal, lung, endometrial, ovarian, and prostate cancers, may be poor. Accordingly, there exists an unmet need for additional treatments for cancer.

### BRIEF SUMMARY OF THE INVENTION

An aspect of the invention provides an isolated or purified T cell receptor (TCR) having antigenic specificity for a human p53^{C135Y}, human p53^{R175H}, or human p53^{M237I} amino acid sequence, wherein the TCR comprises the amino acid sequences of (1) all of SEQ ID NOs: 2-4; (2) all of SEQ ID NOs: 5-7; (3) all of SEQ ID NOs: 2-7; (4) all of SEQ ID NOs: 17-19; (5) all of SEQ ID NOs: 20-22; (6) all of SEQ ID NOs: 17-22; (7) all of SEQ ID NOs: 32-34; (8) all of SEQ ID NOs: 35-37; (9) all of SEQ ID NOs: 32-37; (10) all of SEQ ID NOs: 47-49; (11) all of SEQ ID NOs: 50-52; (12) all of SEQ ID NOs: 47-52; (13) all of SEQ ID NOs: 62-64; (14) all of SEQ ID NOs: 65-67; or (15) all of SEQ ID NOs: 62-67.

Further aspects of the invention provide polypeptides, proteins, nucleic acids, recombinant expression vectors, host cells, populations of cells, and pharmaceutical compositions relating to the TCRs of the invention.

Still further aspects of the invention provide methods of detecting the presence of cancer in a mammal, methods of inducing an immune response against a cancer in a mammal, and methods of treating or preventing cancer in a mammal.

Additional aspects of the invention provide methods of producing a host cell expressing the TCR and methods of producing the TCR, polypeptide, or protein.

### BRIEF DESCRIPTION OF THE SEVERAL VIEWS OF THE DRAWINGS

FIG. 1A is a graph showing the number of IFN-γ spots (per 2e4 cells) measured following co-culture of Patient 4316 TIL from tumor fragment numbers F1-F24 with target cells. Target cells were autologous dendritic cells (DCs) that were (i) pulsed with peptide pools (PP) including p53 C135Y; (ii) transfected with tandem minigene (TMG) RNA encoding p53 C135Y or irrelevant TMG; or (iii) treated with DMSO (control). TIL treated with PMS/ionomycin served as a positive control. Fragment 22, with mutant p53 reactivity, is boxed.

FIG. 1B shows the percentages of TIL expressing 4-1BB and CD4 following co-culture of TILs from patient 4316 with autologous DCs pulsed with DMSO (vehicle), irrelevant peptide KIAA1328 K386R, or the indicated mutant p53-C135Y peptides, as measured by flow cytometry. TIL treated with PMA/ionomycin served as a positive control.

FIG. 1C is a graph showing the percentage of murine TCR constant region-expressing T cells (mTCR⁺) expressing 4-1BB following co-culture of PBLs transduced with the 4316-D TCR with autologous immature DCs pulsed with serially diluted 25-mer peptides p53-C135Y or WT p53-C135.

FIG. 2 shows the percentages of T-cells expressing OX40 and 4-1BB following co-culture of TILs from patient 4141 with target cells following in vitro sensitization (IVS) against p53 R175H (right column) or without IVS (left column), as measured by flow cytometry. Target cells were autologous DCs pulsed with DMSO, the mutant p53 R175H peptide, or the corresponding WT p53 R175 peptide. Activated T cells that upregulated T cell activation markers, 4-1BB and OX40, are outlined in bold. ME, minimal epitope.

FIG. 3A is a graph showing the number of IFN-γ spots (per 2e4 cells) measured following co-culture of Patient 4304 TIL from tumor fragment numbers F1-F24 with target cells. Target cells were autologous DCs that were (i) pulsed with PP including p53 M237I; (ii) transfected with TMG RNA encoding p53 M237I or irrelevant TMG; or (iii) treated with DMSO (control). TIL treated with PMS/ionomycin served as a positive control.

FIG. 3B shows the percentages of CD4⁺ T-cells expressing CD39 and CD103 sorted from tumor digest of Patient 4304, as measured by flow cytometry.

FIG. 3C is a graph showing the number of IFN-γ spots (per 2e4 cells) measured following co-culture of target cells with effector cells. Effector cells were CD4⁺CD103⁺CD39⁺ (squares), CD4⁺CD103⁻CD39⁺ (triangles) or CD4⁺CD103⁻CD39⁻ (circles) cells sorted from the tumor of Patient 4304. Target cells were autologous DCs that were (i) pulsed with PP including p53 M237I; (ii) transfected with TMG RNA encoding p53 M237I or control TMG; or (iii) treated with DMSO (control). TIL cultured alone (TIL only) served as a control.

FIGS. 3D-3F are graphs showing the percentage of murine TCR constant region-expressing, CD3⁺CD4⁺ T cells (mTCR⁺) expressing 4-1BB following co-culture effector cells with autologous immature DCs pulsed with serially diluted 25-mer peptides p53-M237I or WT p53-M237. The effector cells were PBLs independently transduced with a recombinant expression vector encoding 4304 TCR-2 (FIG. 3D), 4304 TCR-4 (FIG. 3E), or 4304 TCR-K (FIG. 3F).

FIGS. 4A-4B show the percentages of 4141 IVS TCR-transduced cells expressing 4-1BB and OX40 following co-culture of 4141 IVS TCR-transduced cells with tumor cell line SK-MEL-5 (4A), SAOS2 (4A), SAOS2 R175H (4A), CEM/C1 (4B), TYK-nu (4B), or KLE (4B). The cell lines are indicated to be positive (+) or negative (−) for p53 R175H and HLA-A*02:01 expression.

FIGS. 5A-5B show an alignment of the amino acid sequences of the nine p53 splice variants. SP|P04637|P53_HUMAN (SEQ ID NO: 1); SP|P04637-2|P53_HUMAN (SEQ ID NO: 81); SP|P04637-3|P53_HUMAN (SEQ ID NO: 82); SP|P04637-4|P53_HUMAN (SEQ ID NO: 83); SP|P04637-5|P53_HUMAN (SEQ ID NO: 84); SP|P04637-6|P53_HUMAN (SEQ ID NO: 85); SP|P04637-7|P53_HUMAN (SEQ ID NO: 86); SP|P04637-8|P53_HUMAN (SEQ ID NO: 87); and SP|P04637-9|P53_HUMAN (SEQ ID NO: 88). The alignment begins with the N-termini shown in FIG. 5A and continues through the C-termini shown in FIG. 5B.

FIG. 6 shows the percentages of 4316-D TCR-transduced cells expressing 4-1BB and OX40 following co-culture of 4316-D TCR-transduced cells with target cells, as measured by flow cytometry. Target cells were 4316 autologous patient-derived xenograft (PDX) tumor cells pulsed with DMSO (left column), WT p53 (middle column), or mutant p53 peptide (right column) in the absence of IFN-γ (top row) or presence of IFN-γ (bottom row).

FIG. 7A is a schematic that illustrates the generation and treatment protocol for preclinical xenograft mice. Female immune-compromised NSG (NOD scid gamma) mice were injected with two million TYK-nu ovarian cells naturally expressing the p53 R175H mutation and HLA-A*02:01. Two weeks later these mice were treated with a vehicle or ten million T cells. Tumor growth was then assessed over the next 30 days.

FIGS. 7B-7C are graphs showing the mean tumor size (mm²) in tumor-bearing mice measured over the course of 30 days following the adoptive cell transfer (ACT) of transduced cells. PBL from two healthy donors (Healthy donor 1 (7B) and Healthy donor 2 (7C)) were independently transduced with the indicated TCR. The mice were in three treatment groups, PBS Vehicle (circle), T cells transduced with irrelevant TCR targeting p53 Y220C (triangle), T cells transduced with with the 4141 IVS TCR (square) with an n of 5. These were compared to mice treated with T cells transduced with the 4141-TCR1a2 (star). The results obtained were consistent with the cells obtained from both healthy donors.

### DETAILED DESCRIPTION OF THE INVENTION

Tumor Protein P53 (also referred to as “TP53” or “p53”) acts as a tumor suppressor by, for example, regulating cell division. The p53 protein is located in the nucleus of the cell, where it binds directly to DNA. When DNA becomes damaged, the p53 protein is involved in determining whether the DNA will be repaired or the damaged cell will undergo apoptosis. If the DNA can be repaired, p53 activates other genes to fix the damage. If the DNA cannot be repaired, the p53 protein prevents the cell from dividing and signals it to undergo apoptosis. By stopping cells with mutated or damaged DNA from dividing, p53 helps prevent the development of tumors. WT (normal) full-length p53 comprises the amino acid sequence of SEQ ID NO: 1.

Mutations in the p53 protein may reduce or eliminate the p53 protein's tumor suppressor function. Alternatively or additionally, a p53 mutation may be a gain-of-function mutation by interfering with WT p53 in a dominant negative fashion. Mutated p53 protein may be expressed in any of a variety of human cancers such as, for example, cholangiocarcinoma, melanoma, colon cancer, rectal cancer, ovarian cancer, endometrial cancer, non-small cell lung cancer (NSCLC), glioblastoma, uterine cervical cancer, head and neck cancer, breast cancer, pancreatic cancer, or bladder cancer.

An aspect of the invention provides an isolated or purified T cell receptor (TCR) having antigenic specificity for a human p53^{C135Y}, human p53^{R175H}, or human p53^{M237I} amino acid sequence (hereinafter, “mutated p53”). Hereinafter, references to a “TCR” also refer to functional portions and functional variants of the TCR, unless specified otherwise. Mutations of p53 are defined herein by reference to the amino acid sequence of full-length, WT p53 (SEQ ID NO: 1). Mutations of p53 are described herein by reference to the amino acid residue present at a particular position, followed by the position number, followed by the amino acid with which that residue has been replaced in the particular mutation under discussion. A p53 amino acid sequence (e.g., a p53 peptide) may comprise fewer than all of the amino acid residues of the full-length, WT p53 protein. Accordingly, the position numbers are defined herein by reference to the WT full-length p53 protein (namely, SEQ ID NO: 1) with the understanding that the actual position of the corresponding residue in a particular example of a p53 amino acid sequence may be different. Because the positions are as defined by SEQ ID NO: 1, the term “C135Y” indicates that the cysteine present at position 135 of SEQ ID NO: 1 is replaced by tyrosine, “R175H” indicates that the arginine present at position 175 of SEQ ID NO: 1 has been replaced with histidine, and “M237I” indicates that the methionine present at position 237 of SEQ ID NO: 1 is replaced by isoleucine. For example, when a particular example of a p53 amino acid sequence is, e.g., TCTYSPALNKMFCQLAKTCPVQLWV (SEQ ID NO: 89) (an exemplary WT p53 peptide corresponding to contiguous amino acid residues 123 to 147 of SEQ ID NO: 1), “C135Y” refers to a substitution of the underlined cysteine in SEQ ID NO: 89 with tyrosine, even though the actual position of the underlined arginine in SEQ ID NO: 89 is 13. Human p53 amino acid sequences with the C135Y mutation are hereinafter referred to as “C135Y” or “p53^{C135Y}.” Human p53 amino acid sequences with the R175H mutation are hereinafter referred to as “R175H” or “p53^{R175H}.” Human p53 amino acid sequences with the M237I mutation are hereinafter referred to as “M237I” or “p53^{M237I}.” As used herein, “mutated p53” refers to human p53^{C135Y}, human p53^{R175H}, or human p53^{M237I}.

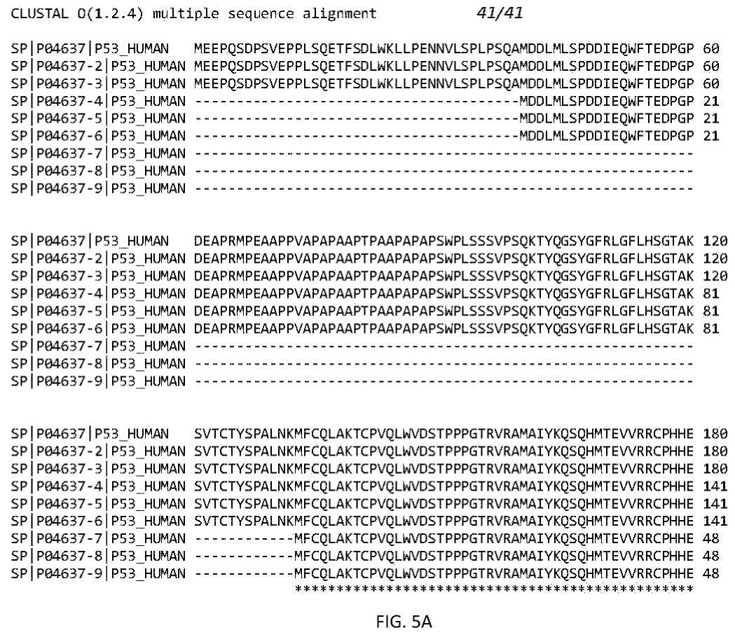

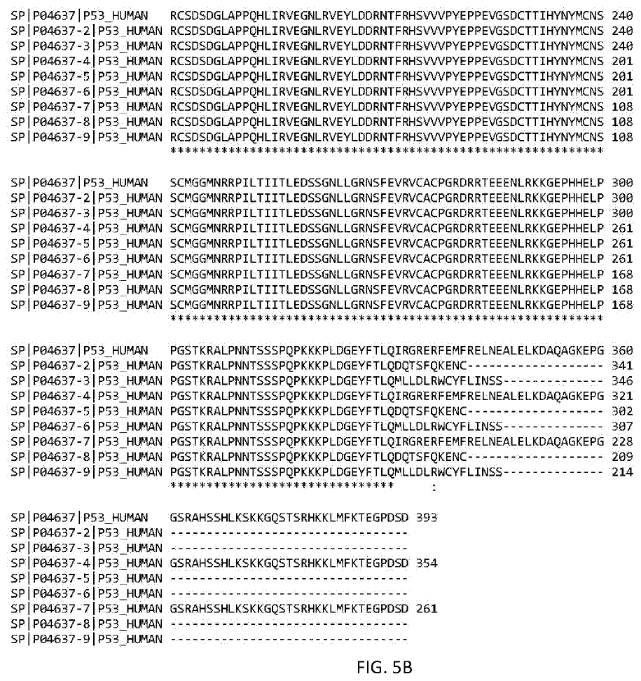

P53 has nine known splice variants. The p53 mutations described herein are conserved over all nine p53 splice variants. An alignment of the nine p53 splice variants is shown in FIG. 5. Accordingly, the inventive TCRs may have antigenic specificity for any mutated p53 amino acid sequence described herein encoded by any of the nine p53 splice variants. Because the positions are as defined by SEQ ID NO: 1, then the actual positions of the amino acid sequence of a particular splice variant of p53 are defined relative to the corresponding positions of SEQ ID NO: 1, and the positions as defined by SEQ ID NO: 1 may be different than the actual positions in a particular splice variant. Thus, for example, mutations refer to a replacement of an amino acid residue in the amino acid sequence of a particular splice variant of p53 corresponding to the indicated position of the 393-amino acid sequence of SEQ ID NO: 1 with the understanding that the actual positions in the splice variant may be different.

In an aspect of the invention, the TCR has antigenic specificity for human p53 with a mutation at position 135, as defined by SEQ ID NO: 1. The p53 mutation at position 135 may be a missense mutation. Accordingly, the mutation at position 135 may be a substitution of the native (WT) cysteine residue present at position 135 with any amino acid residue other than cysteine. In an aspect of the invention, the TCR has antigenic specificity for a human p53^{C135Y} amino acid sequence. For example, the TCR may have antigenic specificity for the human p53^{C135Y} amino acid sequence of TCTYSPALNKMFYQLAKTCPVQLWV (SEQ ID NO: 90). In an aspect of the invention, the TCR does not have antigenic specificity for the wild-type human p53 amino acid sequence of TCTYSPALNKMFCQLAKTCPVQLWV (SEQ ID NO: 89).

In an aspect of the invention, the TCR has antigenic specificity for human p53 with a mutation at position 175, as defined by SEQ ID NO: 1. The p53 mutation at position 175 may be a missense mutation. Accordingly, the mutation at position 175 may be a substitution of the native (WT) arginine residue present at position 175 with any amino acid residue other than arginine. In an aspect of the invention, the TCR has antigenic specificity for a human p53^{R175H} amino acid sequence. For example, the TCR may have antigenic specificity for the human p53^{R175H} amino acid sequence of HMTEVVRHC (SEQ ID NO: 92). In an aspect of the invention, the TCR does not have antigenic specificity for the wild-type human p53 amino acid sequence of HMTEVVRRC (SEQ ID NO: 91).

In an aspect of the invention, the TCR has antigenic specificity for human p53 with a mutation at position 237, as defined by SEQ ID NO: 1. The p53 mutation at position 237 may be a missense mutation. Accordingly, the mutation at position 237 may be a substitution of the native (WT) methionine residue present at position 237 with any amino acid residue other than methionine. In an aspect of the invention, the TCR has antigenic specificity for a human p53^{M237I} amino acid sequence. For example, the TCR may have antigenic specificity for the human p53^{M237I} amino acid sequence of VGSDCTTIHYNYICNSSCMGGMNRR (SEQ ID NO: 94). In an aspect of the invention, the TCR does not have antigenic specificity for the wild-type human p53 amino acid sequence of VGSDCTTIHYNYMCNSSCMGGMNRR (SEQ ID NO: 93).

In an aspect of the invention, the inventive TCRs may be able to recognize mutated p53 in an HLA (human leukocyte antigen)-molecule-dependent manner. “HLA-molecule-dependent manner,” as used herein, means that the TCR elicits an immune response upon binding to mutated p53 within the context of an HLA molecule, which HLA molecule is expressed by the patient from which the TCR was isolated. The inventive TCRs may be able to recognize mutated p53 that is presented by the applicable HLA molecule and may bind to the HLA molecule in addition to mutated p53.

In an aspect of the invention, the inventive TCRs are able to recognize C135Y presented by an HLA Class II molecule. In this regard, the TCR may elicit an immune response upon binding to C135Y within the context of an HLA Class II molecule. The inventive TCRs are able to recognize C135Y that is presented by an HLA Class II molecule and may bind to the HLA Class II molecule in addition to C135Y.

In an aspect of the invention, the inventive TCRs are able to recognize M237I presented by an HLA Class II molecule. In this regard, the TCR may elicit an immune response upon binding to M237I within the context of an HLA Class II molecule. The inventive TCRs are able to recognize M237I that is presented by an HLA Class II molecule and may bind to the HLA Class II molecule in addition to M237I.

In an aspect of the invention, the HLA Class II molecule is an HLA-DR heterodimer. The HLA-DR heterodimer is a cell surface receptor including an α chain and a β chain. The HLA-DR α chain is encoded by the HLA-DRA gene. The HLA-DR β chain is encoded by the HLA-DRB1 gene, the HLA-DRB3 gene, HLA-DRB4 gene, or the HLA-DRB5 gene. Examples of molecules encoded by the HLA-DRB1 gene may include, but are not limited to, HLA-DR1, HLA-DR2, HLA-DR3, HLA-DR4, HLA-DR5, HLA-DR6, HLA-DR7, HLA-DR8, HLA-DR9, HLA-DR10, HLA-DR11, HLA-DR12, HLA-DR13, HLA-DR14, HLA-DR15, HLA-DR16, and HLA-DR17. The HLA-DRB3 gene encodes HLA-DR52. The HLA-DRB4 gene encodes HLA-DR53. The HLA-DRB5 gene encodes HLA-DR51.

In an aspect, the alpha chain of the HLA Class II molecule is expressed by the HLA-DRA1*01:01 allele. In an aspect, the beta chain of the HLA Class II molecule is expressed by the HLA-DRB1*07:01 allele. In an aspect of the invention, the HLA Class II molecule is an HLA-DRB7:HLA-DRA heterodimer. In a preferred aspect, the HLA Class II molecule is a heterodimer of an HLA-DRA1*01:01 chain and an HLA-DRB1*07:01 chain. In an especially preferred aspect, the mutated p53 is C135Y and the HLA Class II molecule is a heterodimer of an HLA-DRA1*01:01 chain and an HLA-DRB1*07:01 chain.

In an aspect, the alpha chain of the HLA Class II molecule is expressed by the HLA-DRA1*01:01 allele. In an aspect, the beta chain of the HLA Class II molecule is expressed by the HLA-DRB1*01:01 allele. In an aspect of the invention, the HLA Class II molecule is an HLA-DRB1:HLA-DRA heterodimer. In a preferred aspect, the HLA Class II molecule is a heterodimer of an HLA-DRA1*01:01 chain and an HLA-DRB1*01:01 chain. In an especially preferred aspect, the mutated p53 is M237I and the HLA Class II molecule is a heterodimer of an HLA-DRA1*01:01 chain and an HLA-DRB1*01:01 chain.

In an aspect of the invention, the inventive TCRs are able to recognize R175H presented by an HLA Class I molecule. In this regard, the TCR may elicit an immune response upon binding to R175H within the context of an HLA Class I molecule. The inventive TCRs are able to recognize R175H that is presented by an HLA Class I molecule and may bind to the HLA Class I molecule in addition to R175H.

In an embodiment of the invention, the HLA Class I molecule is an HLA-A molecule. The HLA-A molecule is a heterodimer of an α chain and β2 microglobulin. The HLA-A α chain may be encoded by an HLA-A gene. β2 microglobulin binds non-covalently to the alpha1, alpha2 and alpha3 domains of the alpha chain to build the HLA-A complex. The HLA-A molecule may be any HLA-A molecule. In an embodiment of the invention, the HLA Class I molecule is an HLA-A2 molecule. The HLA-A2 molecule may be any HLA-A2 molecule. Examples of HLA-A2 molecules may include, but are not limited to, those encoded by the HLA-A*02:01, HLA-A*02:02, HLA-A*02:03 allele, HLA-A*02:05, HLA-A*02:06, HLA-A*02:07 allele, or HLA-A*02:11 allele. Preferably, the HLA Class I molecule is encoded by the HLA-A*02:01 allele.

The TCRs of the invention may provide any one or more of many advantages, including when expressed by cells used for adoptive cell transfer. Mutated p53 is expressed by cancer cells and is not expressed by normal, noncancerous cells. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. Moreover, the inventive TCRs may, advantageously, successfully treat or prevent mutated p53-positive cancers that do not respond to other types of treatment such as, for example, chemotherapy, surgery, or radiation. Additionally, the inventive TCRs may provide highly avid recognition of mutated p53, which may provide the ability to recognize unmanipulated tumor cells (e.g., tumor cells that have not been treated with interferon (IFN)-γ, transfected with a vector encoding one or both of mutated p53 and the applicable HLA molecule, pulsed with a p53 peptide with the p53 mutation, or a combination thereof). Mutations in p53 are common across different tumor types. Roughly half of all tumors harbor a mutation in p53, about half of which will be a missense mutation. The R175H mutation is common, affecting about 5% of all patients with solid cancers. The C135Y and M237I mutations are also highly recurrent, each of which affects about 0.4% of all cancer patients. Accordingly, the inventive TCRs may increase the number of patients who may be eligible for treatment with immunotherapy.

The phrase “antigenic specificity,” as used herein, means that the TCR can specifically bind to and immunologically recognize mutated p53 with high avidity. For example, a TCR may be considered to have “antigenic specificity” for mutated p53 if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with (a) antigen-negative, applicable HLA molecule positive target cells pulsed with mutated p53 peptide (e.g., about 0.1 ng/mL to about 10,000 ng/mL, 0.1 ng/mL, 0.5 ng/mL, 1 ng/mL, 5 ng/mL, 10 ng/mL, 100 ng/mL, 500 ng/mL, 1,000 ng/mL, 5,000 ng/mL, 10,000 ng/mL, or a range defined by any two of the foregoing values) or (b) antigen-negative, applicable HLA molecule positive target cells into which a nucleotide sequence encoding mutated p53 has been introduced such that the target cell expresses mutated p53. Cells expressing the inventive TCRs may also secrete IFN-γ upon co-culture with antigen-negative, applicable HLA molecule positive target cells pulsed with higher concentrations of mutated p53 peptide.

Alternatively or additionally, a TCR may be considered to have “antigenic specificity” for mutated p53 if T cells expressing the TCR secrete at least twice as much IFN-γ upon co-culture with (a) antigen-negative, applicable HLA molecule positive target cells pulsed with mutated p53 peptide or (b) antigen-negative, applicable HLA molecule positive target cells into which a nucleotide sequence encoding mutated p53 has been introduced such that the target cell expresses mutated p53 as compared to the amount of IFN-γ expressed by a negative control. The negative control may be, for example, (i) T cells expressing the TCR, co-cultured with (a) antigen-negative, applicable HLA molecule positive target cells pulsed with the same concentration of an irrelevant peptide (e.g., some other peptide with a different sequence from the mutated p53 peptide) or (b) antigen-negative, applicable HLA molecule positive target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative, applicable HLA molecule positive target cells pulsed with the same concentration of mutated p53 peptide or (b) antigen-negative, applicable HLA molecule positive target cells into which a nucleotide sequence encoding mutated p53 has been introduced such that the target cell expresses mutated p53. IFN-γ secretion may be measured by methods known in the art such as, for example, enzyme-linked immunosorbent assay (ELISA). The concentration of pulsed peptide may be as described herein with respect to other aspects of the invention.

Alternatively or additionally, a TCR may be considered to have “antigenic specificity” for mutated p53 if at least twice as many of the numbers of T cells expressing the TCR secrete IFN-γ upon co-culture with (a) antigen-negative, applicable HLA molecule positive target cells pulsed with mutated p53 peptide or (b) antigen-negative, applicable HLA molecule positive target cells into which a nucleotide sequence encoding mutated p53 has been introduced such that the target cell expresses mutated p53 as compared to the numbers of negative control T cells that secrete IFN-γ. The concentration of peptide and the negative control may be as described herein with respect to other aspects of the invention. The numbers of cells secreting IFN-γ may be measured by methods known in the art such as, for example, enzyme-linked immunospot (ELISOT) assay.

Alternatively or additionally, a TCR may be considered to have “antigenic specificity” for mutated p53 if at least twice as many spots are detected by ELISPOT for the T cells expressing the TCR upon co-culture with (a) antigen-negative, applicable HLA molecule positive target cells pulsed with mutated p53 peptide or (b) antigen-negative, applicable HLA molecule positive target cells into which a nucleotide sequence encoding mutated p53 has been introduced such that the target cell expresses mutated p53 as compared to the number of spots detected by ELISPOT for negative control T cells co-cultured with the same target cells. The concentration of peptide and the negative control may be as described herein with respect to other aspects of the invention.

Alternatively or additionally, a TCR may be considered to have “antigenic specificity” for mutated p53 if T cells expressing the TCR upregulate expression of one or both of 4-1BB and OX40 as measured by, for example, flow cytometry after stimulation with target cells expressing mutated p53.

An aspect of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for mutated p53.

In an aspect of the invention, the TCR comprises two polypeptide chains, each of which comprises a variable region comprising a complementarity determining region (CDR)1, a CDR2, and a CDR3 of a TCR. In an aspect of the invention, the TCR comprises a first polypeptide chain comprising an α chain CDR1 (CDR1α), an α chain CDR2 (CDR2α), and an α chain CDR3 (CDR3α), and a second polypeptide chain comprising a β chain CDR1 (CDR1β), a β chain CDR2 (CDR2β), and a β chain CDR3 (CDR3β). In an aspect of the invention, the TCR comprises the amino acid sequences of (1) all of SEQ ID NOs: 2-7 (4316-D TCR); (2) all of SEQ ID NOs: 17-22 (4141 IVS TCR); (3) all of SEQ ID NOs: 32-37 (4304 TCR-2); (4) all of SEQ ID NOs: 47-52 (4304 TCR-4); or (5) all of SEQ ID NOs: 62-67 (4304 TCR-K). Each one of the foregoing five collections of amino acid sequences in this paragraph sets forth the six CDR regions of each of five different TCRs having antigenic specificity for mutated human p53. The six amino acid sequences in each collection correspond to the CDR1α, CDR2α, CDR3α, CDR1β, CDR2β, and CDR3β of a TCR, respectively.

The TCR may comprise the amino acid sequences of any one or more of: SEQ ID NOs: 2-7, 17-22, 32-37, 47-52, and 62-67. In an aspect of the invention, the TCR comprises an isolated or purified TCR having antigenic specificity for a human p53^{C135Y}, human p53^{R175H}, or human p53^{M237I} amino acid sequence, wherein the TCR comprises the amino acid sequences of: (1) all of SEQ ID NOs: 2-4; (2) all of SEQ ID NOs: 5-7; (3) all of SEQ ID NOs: 2-7; (4) all of SEQ ID NOs: 17-19; (5) all of SEQ ID NOs: 20-22; (6) all of SEQ ID NOs: 17-22; (7) all of SEQ ID NOs: 32-34; (8) all of SEQ ID NOs: 35-37; (9) all of SEQ ID NOs: 32-37; (10) all of SEQ ID NOs: 47-49; (11) all of SEQ ID NOs: 50-52; (12) all of SEQ ID NOs: 47-52; (13) all of SEQ ID NOs: 62-64; (14) all of SEQ ID NOs: 65-67; or (15) all of SEQ ID NOs: 62-67.

In an aspect of the invention, the TCR comprises an α chain variable region amino acid sequence and a β chain variable region amino acid sequence, which together comprise one of the collections of CDRs set forth above. In this regard, the TCR can comprise the amino acid sequences of (1) both of SEQ ID NOs: 8 and 9; (2) both of SEQ ID NOs: 10 and 11; (3) both of SEQ ID NOs: 23 and 24; (4) both of SEQ ID NOs: 25 and 26; (5) both of SEQ ID NOs: 38 and 39; (6) both of SEQ ID NOs: 40 and 41; (7) both of SEQ ID NOs: 53 and 54; (8) both of SEQ ID NOs: 55 and 56; (9) both of SEQ ID NOs: 68 and 69; or (10) both of SEQ ID NOs: 70 and 71. Each one of the foregoing collections of amino acid sequences in this paragraph sets forth the two variable regions of each of five different TCRs having antigenic specificity for mutated human p53. The two amino acid sequences in each collection correspond to the variable region of the α chain and the variable region of the β chain of a TCR, respectively.

The TCR may, e.g., comprise the amino acid sequence of any one or more of SEQ ID NOs: 8, 9, 10, 11, 23, 24, 25, 26, 38, 39, 40, 41, 53, 54, 55, 56, 68, 69, 70, and 71. In an aspect of the invention, the TCR comprises the amino acid sequence(s) of: (1) SEQ ID NO: 8; (2) SEQ ID NO: 9; (3) both of SEQ ID NOs: 8 and 9; (4) SEQ ID NO: 10; (5) SEQ ID NO: 11; (6) both of SEQ ID NOs: 10 and 11; (7) SEQ ID NO: 23; (8) SEQ ID NO: 24; (9) both of SEQ ID NOs: 23 and 24; (10) SEQ ID NO: 25; (11) SEQ ID NO: 26; (12) both of SEQ ID NOs: 25 and 26; (13) SEQ ID NO: 38; (14) SEQ ID NOs: 39; (15) both of SEQ ID NOs: 38 and 39; (16) SEQ ID NO: 40; (17) SEQ ID NO: 41; (18) both of SEQ ID NOs: 40 and 41; (19) SEQ ID NO: 53; (20) SEQ ID NO: 54; (21) both of SEQ ID NOs: 53 and 54; (22) SEQ ID NO: 55; (23) SEQ ID NO: 56; (24) both of SEQ ID NOs: 55 and 56; (25) SEQ ID NO: 68; (26) SEQ ID NO: 69; (27) both of SEQ ID NOs: 68 and 69; (28) SEQ ID NO: 70; (29) SEQ ID NO: 71; or (30) both of SEQ ID NOs: 70 and 71.

The inventive TCRs may further comprise a constant region. The constant region may be derived from any suitable species such as, e.g., human or mouse. In an aspect of the invention, the TCRs further comprise a murine constant region. As used herein, the term “murine” or “human,” when referring to a TCR or any component of a TCR described herein (e.g., complementarity determining region (CDR), variable region, constant region, alpha chain, and/or beta chain), means a TCR (or component thereof) which is derived from a mouse or a human, respectively, i.e., a TCR (or component thereof) that originated from or was, at one time, expressed by a mouse T cell or a human T cell, respectively. In an aspect of the invention, the TCR may comprise a murine α chain constant region and a murine β chain constant region. The murine α chain constant region may be modified or unmodified. A modified murine α chain constant region may be, e.g., cysteine-substituted, LVL-modified, or both cysteine-substituted and LVL-modified, as described, for example, in U.S. Pat. No. 10,174,098. The murine β chain constant region may be modified or unmodified. A modified murine β chain constant region may be, e.g., cysteine-substituted, as described, for example, in U.S. Pat. No. 10,174,098. In an aspect of the invention, the TCR comprises a cysteine-substituted, LVL-modified murine α chain constant region comprising the amino acid sequence of SEQ ID NO: 77 or 78. In an aspect of the invention, the TCR comprises a cysteine-substituted murine β chain constant region comprising the amino acid sequence of SEQ ID NO: 79.

In an aspect of the invention, the inventive TCR can comprise an α chain of a TCR and a β chain of a TCR. The α chain of the TCR may comprise a variable region of an α chain and a constant region of an α chain. An α chain of this type can be paired with any 3 chain of a TCR. The β chain may comprise a variable region of a β chain and a constant region of a β chain.

In some aspects, the amino acid sequence of any of the α chains and/or β chains disclosed herein further comprises the amino acid sequence RAKR (SEQ ID NO: 95) at the C-terminal end.

In an aspect of the invention, the TCR can comprise the amino acid sequences of: (1) both of SEQ ID NOs: 12 and 13; (2) both of SEQ ID NOs: 14 and 15; (3) both of SEQ ID NOs: 27 and 28; (4) both of SEQ ID NOs: 29 and 30; (5) both of SEQ ID NOs: 42 and 43; (6) both of SEQ ID NOs: 44 and 45; (7) both of SEQ ID NOs: 57 and 58; (8) both of SEQ ID NOs: 59 and 60; (9) both of SEQ ID NOs: 72 and 73; or (10) both of SEQ ID NOs: 74 and 75. Each one of the foregoing collections of amino acid sequences in this paragraph sets forth the α chain and β chain of each of five different TCRs having antigenic specificity for mutated human p53. The two amino acid sequences in each collection correspond to the α chain and the β chain of a TCR, respectively.

The TCR may comprise the amino acid sequence of any one or more of SEQ ID NOs: 12, 13, 14, 15, 27, 28, 29, 30, 42, 43, 44, 45, 57, 58, 59, 60, 72, 73, 74, and 75. In an aspect of the invention, the TCR comprises the amino acid sequences of (1) SEQ ID NO: 12; (2) SEQ ID NO: 13; (3) both of SEQ ID NOs: 12 and 13; (4) SEQ ID NO: 14; (5) SEQ ID NO: 15; (6) both of SEQ ID NOs: 14 and 15; (7) SEQ ID NO: 27; (8) SEQ ID NO: 28; (9) both of SEQ ID NOs: 27 and 28; (10) SEQ ID NO: 29; (11) SEQ ID NO: 30; (12) both of SEQ ID NOs: 29 and 30; (13) SEQ ID NO: 42; (14) SEQ ID NO: 43; (15) both of SEQ ID NOs: 42 and 43; (16) SEQ ID NO: 44; (17) SEQ ID NO: 45; (18) both of SEQ ID NOs: 44 and 45; (19) SEQ ID NO: 57; (20) SEQ ID NO: 58; (21) both of SEQ ID NOs: 57 and 58; (22) SEQ ID NO: 59; (23) SEQ ID NO: 60; (24) both of SEQ ID NOs: 59 and 60; (25) SEQ ID NO: 72; (26) SEQ ID NO: 73; (27) both of SEQ ID NOs: 72 and 73; (28) SEQ ID NO: 74; (29) SEQ ID NO: 75; or (30) both of SEQ ID NOs: 74 and 75.

Included in the scope of the invention are functional variants of the inventive TCRs described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to mutated p53 for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, at least about 50%, at least about 75%, at least about 80%, at least about 90%, at least about 95%, at least about 96%, at least about 97%, at least about 98%, at least about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein.

Also provided by the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to mutated p53. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to mutated p53 (e.g., in an applicable HLA molecule-dependent manner), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to mutated p53; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs of the invention, such as a functional portion comprising one of more of CDR1, CDR2, and CDR3 of the variable region(s) of the α chain and/or β chain of a TCR of the invention. In an aspect of the invention, the polypeptide can comprise the amino acid sequences of (1) all of SEQ ID NOs: 2-7; (2) all of SEQ ID NOs: 17-22; (3) all of SEQ ID NOs: 32-37; (4) all of SEQ ID NOs: 47-52; or (5) all of SEQ ID NOs: 62-67. The polypeptide may comprise the amino acid sequences of any one or more of SEQ ID NOs: 2-7, 17-22, 32-37, 47-52, and 62-67. In an aspect of the invention, the polypeptide comprises the amino acid sequences of: (1) all of SEQ ID NOs: 2-4; (2) all of SEQ ID NOs: 5-7; (3) all of SEQ ID NOs: 2-7; (4) all of SEQ ID NOs: 17-19; (5) all of SEQ ID NOs: 20-22; (6) all of SEQ ID NOs: 17-22; (7) all of SEQ ID NOs: 32-34; (8) all of SEQ ID NOs: 35-37; (9) all of SEQ ID NOs: 32-37; (10) all of SEQ ID NOs: 47-49; (11) all of SEQ ID NOs: 50-52; (12) all of SEQ ID NOs: 47-52; (13) all of SEQ ID NOs: 62-64; (14) all of SEQ ID NOs: 65-67; or (15) all of SEQ ID NOs: 62-67.

In an aspect of the invention, the inventive polypeptide can comprise, for instance, the variable region of the inventive TCR comprising a combination of the CDR regions set forth above. In this regard, the polypeptide can comprise, e.g., the amino acid sequences of: (1) both of SEQ ID NOs: 8 and 9; (2) both of SEQ ID NOs: 10 and 11; (3) both of SEQ ID NOs: 23 and 24; (4) both of SEQ ID NOs: 25 and 26; (5) both of SEQ ID NOs: 38 and 39; (6) both of SEQ ID NOs: 40 and 41; (7) both of SEQ ID NOs: 53 and 54; (8) both of SEQ ID NOs: 55 and 56; (9) both of SEQ ID NOs: 68 and 69; or (10) both of SEQ ID NOs: 70 and 71. The polypeptide may, e.g., comprise the amino acid sequence of any one or more of SEQ ID NOs: 8, 9, 10, 11, 23, 24, 25, 26, 38, 39, 40, 41, 53, 54, 55, 56, 68, 69, 70, and 71. In an aspect of the invention, the polypeptide comprises the amino acid sequence(s) of (1) SEQ ID NO: 8; (2) SEQ ID NO: 9; (3) both of SEQ ID NOs: 8 and 9; (4) SEQ ID NO: 10; (5) SEQ ID NO: 11; (6) both of SEQ ID NOs: 10 and 11; (7) SEQ ID NO: 23; (8) SEQ ID NO: 24; (9) both of SEQ ID NOs: 23 and 24; (10) SEQ ID NO: 25; (11) SEQ ID NO: 26; (12) both of SEQ ID NOs: 25 and 26; (13) SEQ ID NO: 38; (14) SEQ ID NOs: 39; (15) both of SEQ ID NOs: 38 and 39; (16) SEQ ID NO: 40; (17) SEQ ID NO: 41; (18) both of SEQ ID NOs: 40 and 41; (19) SEQ ID NO: 53; (20) SEQ ID NO: 54; (21) both of SEQ ID NOs: 53 and 54; (22) SEQ ID NO: 55; (23) SEQ ID NO: 56; (24) both of SEQ ID NOs: 55 and 56; (25) SEQ ID NO: 68; (26) SEQ ID NO: 69; (27) both of SEQ ID NOs: 68 and 69; (28) SEQ ID NO: 70; (29) SEQ ID NO: 71; or (30) both of SEQ ID NOs: 70 and 71.

In an aspect of the invention, the inventive polypeptide can further comprise the constant region of the inventive TCR set forth above. In this regard, the polypeptide can comprise, e.g., the amino acid sequence of (i) one of SEQ ID NOs 77-79 or (ii) SEQ ID NO: 79 and one of SEQ ID NOs: 77 and 78.

In an aspect of the invention, the inventive polypeptide may comprise an α chain and a β chain of the inventive TCR. In this regard, the polypeptide can comprise, e.g., the amino acid sequences of (1) both of SEQ ID NOs: 12 and 13; (2) both of SEQ ID NOs: 14 and 15; (3) both of SEQ ID NOs: 27 and 28; (4) both of SEQ ID NOs: 29 and 30; (5) both of SEQ ID NOs: 42 and 43; (6) both of SEQ ID NOs: 44 and 45; (7) both of SEQ ID NOs: 57 and 58; (8) both of SEQ ID NOs: 59 and 60; (9) both of SEQ ID NOs: 72 and 73; or (10) both of SEQ ID NOs: 74 and 75. The polypeptide may comprise the amino acid sequence of any one or more of SEQ ID NOs: 12, 13, 14, 15, 27, 28, 29, 30, 42, 43, 44, 45, 57, 58, 59, 60, 72, 73, 74, and 75. In an aspect of the invention, the polypeptide comprises the amino acid sequences of: (1) SEQ ID NO: 12; (2) SEQ ID NO: 13; (3) both of SEQ ID NOs: 12 and 13; (4) SEQ ID NO: 14; (5) SEQ ID NO: 15; (6) both of SEQ ID NOs: 14 and 15; (7) SEQ ID NO: 27; (8) SEQ ID NO: 28; (9) both of SEQ ID NOs: 27 and 28; (10) SEQ ID NO: 29; (11) SEQ ID NO: 30; (12) both of SEQ ID NOs: 29 and 30; (13) SEQ ID NO: 42; (14) SEQ ID NO: 43; (15) both of SEQ ID NOs: 42 and 43; (16) SEQ ID NO: 44; (17) SEQ ID NO: 45; (18) both of SEQ ID NOs: 44 and 45; (19) SEQ ID NO: 57; (20) SEQ ID NO: 58; (21) both of SEQ ID NOs: 57 and 58; (22) SEQ ID NO: 59; (23) SEQ ID NO: 60; (24) both of SEQ ID NOs: 59 and 60; (25) SEQ ID NO: 72; (26) SEQ ID NO: 73; (27) both of SEQ ID NOs: 72 and 73; (28) SEQ ID NO: 74; (29) SEQ ID NO: 75; or (30) both of SEQ ID NOs: 74 and 75.

An aspect of the invention further provides a protein comprising at least one of the polypeptides described herein. By “protein” is meant a molecule comprising one or more polypeptide chains. In an aspect, the protein of the invention can comprise: first and second polypeptide chains, wherein: (1) the first polypeptide chain comprises the amino acid sequences of all of SEQ ID NOs: 2-4; (2) the second polypeptide chain comprises the amino acid sequences of all of SEQ ID NOs: 5-7; (3) the first polypeptide chain comprises the amino acid sequences of all of SEQ ID NOs: 2-4 and the second polypeptide chain comprises the amino acid sequences of all of SEQ ID NOs: 5-7; (4) the first polypeptide chain comprises the amino acid sequences of all of SEQ ID NOs: 17-19; (5) the second polypeptide chain comprises the amino acid sequences of all of SEQ ID NOs: 20-22; (6) the first polypeptide chain comprises the amino acid sequences of all of SEQ ID NOs: 17-19 and the second polypeptide chain comprises the amino acid sequences of all of SEQ ID NOs: 20-22; (7) the first polypeptide chain comprises the amino acid sequences of all of SEQ ID NOs: 32-34; (8) the second polypeptide chain comprises the amino acid sequences of all of SEQ ID NOs: 35-37; (9) the first polypeptide chain comprises the amino acid sequences of all of SEQ ID NOs: 32-34 and the second polypeptide chain comprises the amino acid sequences of all of SEQ ID NOs: 35-37; (10) the first polypeptide chain comprises the amino acid sequences of all of SEQ ID NOs: 47-49; (11) the second polypeptide chain comprises the amino acid sequences of all of SEQ ID NOs: 50-52; (12) the first polypeptide chain comprises the amino acid sequences of all of SEQ ID NOs: 47-49 and the second polypeptide chain comprises the amino acid sequences of all of SEQ ID NOs: 50-52; (13) the first polypeptide chain comprises the amino acid sequences of all of SEQ ID NOs: 62-64; (14) the second polypeptide chain comprises the amino acid sequences of all of SEQ ID NOs: 65-67; or (15) the first polypeptide chain comprises the amino acid sequences of all of SEQ ID NOs: 62-64 and the second polypeptide chain comprises the amino acid sequences of all of SEQ ID NOs: 65-67.

In an aspect of the invention, the protein comprises first and second polypeptide chains, wherein: (1) the first polypeptide chain comprises the amino acid sequence of SEQ ID NO: 8; (2) the second polypeptide chain comprises the amino acid sequence of SEQ ID NO: 9; (3) the first polypeptide chain comprises the amino acid sequence of SEQ ID NO: 8 and the second polypeptide chain comprises the amino acid sequence of SEQ ID NO: 9; (4) the first polypeptide chain comprises the amino acid sequence of SEQ ID NO: 10; (5) the second polypeptide chain comprises the amino acid sequence of SEQ ID NO: 11; (6) the first polypeptide chain comprises the amino acid sequence of SEQ ID NO: 10 and the second polypeptide chain comprises the amino acid sequence of SEQ ID NO: 11; (7) the first polypeptide chain comprises the amino acid sequence of SEQ ID NO: 23; (8) the second polypeptide chain comprises the amino acid sequence of SEQ ID NO: 24; (9) the first polypeptide chain comprises the amino acid sequence of SEQ ID NO: 23 and the second polypeptide chain comprises the amino acid sequence of SEQ ID NO: 24; (10) the first polypeptide chain comprises the amino acid sequence of SEQ ID NO: 25; (11) the second polypeptide chain comprises the amino acid sequence of SEQ ID NO: 26; (12) the first polypeptide chain comprises the amino acid sequence of SEQ ID NO: 25 and the second polypeptide chain comprises the amino acid sequence of SEQ ID NO: 26; (13) the first polypeptide chain comprises the amino acid sequence of SEQ ID NO: 38; (14) the second polypeptide chain comprises the amino acid sequence of SEQ ID NO: 39; (15) the first polypeptide chain comprises the amino acid sequence of SEQ ID NO: 38 and the second polypeptide chain comprises the amino acid sequence of SEQ ID NO: 39; (16) the first polypeptide chain comprises the amino acid sequence of SEQ ID NO: 40; (17) the second polypeptide chain comprises the amino acid sequence of SEQ ID NO: 41; (18) the first polypeptide chain comprises the amino acid sequence of SEQ ID NO: 40 and the second polypeptide chain comprises the amino acid sequence of SEQ ID NO: 41; (19) the first polypeptide chain comprises the amino acid sequence of SEQ ID NO: 53; (20) the second polypeptide chain comprises the amino acid sequence of SEQ ID NO: 54; (21) the first polypeptide chain comprises the amino acid sequence of SEQ ID NO: 53 and the second polypeptide chain comprises the amino acid sequence of SEQ ID NO: 54; (22) the first polypeptide chain comprises the amino acid sequence of SEQ ID NO: 55; (23) the second polypeptide chain comprises the amino acid sequence of SEQ ID NO: 56; (24) the first polypeptide chain comprises the amino acid sequence of SEQ ID NO: 55 and the second polypeptide chain comprises the amino acid sequence of SEQ ID NO: 56; (25) the first polypeptide chain comprises the amino acid sequence of SEQ ID NO: 68; (26) the second polypeptide chain comprises the amino acid sequence of SEQ ID NO: 69; (27) the first polypeptide chain comprises the amino acid sequence of SEQ ID NO: 68 and the second polypeptide chain comprises the amino acid sequence of SEQ ID NO: 69; (28) the first polypeptide chain comprises the amino acid sequence of SEQ ID NO: 70; (29) the second polypeptide chain comprises the amino acid sequence of SEQ ID NO: 71; or (30) the first polypeptide chain comprises the amino acid sequence of SEQ ID NO: 70 and the second polypeptide chain comprises the amino acid sequence of SEQ ID NO: 71.

In an aspect of the invention, the protein comprises the protein comprises first and second polypeptide chains, wherein: (1) the first polypeptide chain comprises the amino acid sequence of SEQ ID NO: 12; (2) the second polypeptide chain comprises the amino acid sequence of SEQ ID NO: 13; (3) the first polypeptide chain comprises the amino acid sequence of SEQ ID NO: 12 and the second polypeptide chain comprises the amino acid sequence of SEQ ID NO: 13; (4) the first polypeptide chain comprises the amino acid sequence of SEQ ID NO: 14; (5) the second polypeptide chain comprises the amino acid sequence of SEQ ID NO: 15; (6) the first polypeptide chain comprises the amino acid sequence of SEQ ID NO: 14 and the second polypeptide chain comprises the amino acid sequence of SEQ ID NO: 15; (7) the first polypeptide chain comprises the amino acid sequence of SEQ ID NO: 27; (8) the second polypeptide chain comprises the amino acid sequence of SEQ ID NO: 28; (9) the first polypeptide chain comprises the amino acid sequence of SEQ ID NO: 27 and the second polypeptide chain comprises the amino acid sequence of SEQ ID NO: 28; (10) the first polypeptide chain comprises the amino acid sequence of SEQ ID NO: 29; (11) the second polypeptide chain comprises the amino acid sequence of SEQ ID NO: 30; (12) the first polypeptide chain comprises the amino acid sequence of SEQ ID NO: 29 and the second polypeptide chain comprises the amino acid sequence of SEQ ID NO: 30; (13) the first polypeptide chain comprises the amino acid sequence of SEQ ID NO: 42; (14) the second polypeptide chain comprises the amino acid sequence of SEQ ID NO: 43; (15) the first polypeptide chain comprises the amino acid sequence of SEQ ID NO: 42 and the second polypeptide chain comprises the amino acid sequence of SEQ ID NO: 43; (16) the first polypeptide chain comprises the amino acid sequence of SEQ ID NO: 44; (17) the second polypeptide chain comprises the amino acid sequence of SEQ ID NO: 45; (18) the first polypeptide chain comprises the amino acid sequence of SEQ ID NO: 44 and the second polypeptide chain comprises the amino acid sequence of SEQ ID NO: 45; (19) the first polypeptide chain comprises the amino acid sequence of SEQ ID NO: 57; (20) the second polypeptide chain comprises the amino acid sequence of SEQ ID NO: 58; (21) the first polypeptide chain comprises the amino acid sequence of SEQ ID NO: 57 and the second polypeptide chain comprises the amino acid sequence of SEQ ID NO: 58; (22) the first polypeptide chain comprises the amino acid sequence of SEQ ID NO: 59; (23) the second polypeptide chain comprises the amino acid sequence of SEQ ID NO: 60; (24) the first polypeptide chain comprises the amino acid sequence of SEQ ID NO: 59 and the second polypeptide chain comprises the amino acid sequence of SEQ ID NO: 60; (25) the first polypeptide chain comprises the amino acid sequence of SEQ ID NO: 72; (26) the second polypeptide chain comprises the amino acid sequence of SEQ ID NO: 73; (27) the first polypeptide chain comprises the amino acid sequence of SEQ ID NO: 72 and the second polypeptide chain comprises the amino acid sequence of SEQ ID NO: 73; (28) the first polypeptide chain comprises the amino acid sequence of SEQ ID NO: 74; (29) the second polypeptide chain comprises the amino acid sequence of SEQ ID NO: 75; or (30) the first polypeptide chain comprises the amino acid sequence of SEQ ID NO: 74 and the second polypeptide chain comprises the amino acid sequence of SEQ ID NO: 75.

The protein of the invention may be a TCR. Alternatively, if the first and/or second polypeptide chain(s) of the protein further comprise(s) other amino acid sequences, e.g., an amino acid sequence encoding an immunoglobulin or a portion thereof, then the inventive protein can be a fusion protein. In this regard, an aspect of the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some aspects of the invention, the TCRs, polypeptides, and proteins of the invention may be expressed as a single protein comprising a linker peptide linking the α chain and the β chain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. For example, the linker peptide may comprise the amino acid sequence of RAKRSGSGATNFSLLKQAGDVEENPGP (SEQ ID NO: 80). Upon expression of the construct including the linker peptide by a host cell, the linker peptide may be cleaved, resulting in separated α and β chains. In an aspect of the invention, the TCR, polypeptide, or protein may comprise an amino acid sequence comprising a full-length α chain, a full-length β chain, and a linker peptide positioned between the α and β chains.

In some aspects, the TCR, polypeptide or protein disclosed herein comprises an α chain and/or a β chain, as disclosed herein, comprising a signal peptide. In some aspects, the sequence of the signal peptide of any of the α chains and/or β chains disclosed herein comprises an leucine, lysine, alanine or histidine residue substituted for the wild-type residue at position 2.

In some aspects, the TCR, polypeptide or protein disclosed herein comprises a mature version of an α chain and/or a β chain, as disclosed herein, that lacks a signal peptide.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

The TCRs, polypeptides, and proteins of the invention can be of any length, i.e., can comprise any number of amino acids, provided that the TCRs, polypeptides, or proteins retain their biological activity, e.g., the ability to specifically bind to mutated p53; detect cancer in a mammal; or treat or prevent cancer in a mammal, etc. For example, the polypeptide can be in the range of from about 50 to about 5000 amino acids long, such as 50, 70, 75, 100, 125, 150, 175, 200, 300, 400, 500, 600, 700, 800, 900, 1000 or more amino acids in length. In this regard, the polypeptides of the invention also include oligopeptides.

The TCRs, polypeptides, and proteins of the invention of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs, polypeptides, and proteins of the invention can be, e.g., glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be synthesized by any of a variety of commercial entities. In this respect, the inventive TCRs, polypeptides, and proteins can be synthetic, recombinant, isolated, and/or purified.

An aspect of the invention provides a nucleic acid comprising a nucleotide sequence encoding any of the TCRs, polypeptides, or proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In an aspect, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

An aspect of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 8 and 9; 9 and 8; 10 and 11; 11 and 10; 12 and 13; 13 and 12; 14 and 15; 15 and 14; 23 and 24; 24 and 23; 25 and 26; 26 and 25; 27 and 28; 28 and 27; 29 and 30; 30 and 29; 38 and 39; 39 and 38; 40 and 41; 41 and 40; 42 and 43; 43 and 42; 44 and 45; 45 and 44; 53 and 54; 54 and 53; 55 and 56; 56 and 55; 57 and 58; 58 and 57; 59 and 60; 60 and 59; 68 and 69; 69 and 68; 70 and 71; 71 and 70; 72 and 73; 73 and 72; 74 and 75; or 75 and 74.

In an aspect of the invention, the nucleic acid further comprises a third nucleotide acid sequence interposed between the first and second nucleotide sequence, wherein the third nucleotide sequence encodes a cleavable linker peptide. For example, the cleavable linker peptide may comprise the amino acid sequence of RAKRSGSGATNFSLLKQAGDVEENPGP (SEQ ID NO: 80). In an aspect, of the invention, nucleic acid encodes an amino acid sequence selected from the group consisting of SEQ ID NO: 16, 31, 46, 61, and 76.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be synthesized by any of a variety of commercial entities.

In an aspect of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

An aspect of the invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

An aspect of the invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

The nucleic acids of the invention can be incorporated into a recombinant expression vector. In this regard, an aspect of the invention provides a recombinant expression vector comprising any of the nucleic acids of the invention. In an aspect of the invention, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the transposon/transposase series, pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech). Preferably, the recombinant expression vector is a transposon or a viral vector, e.g., a lentiviral vector or a retroviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter, e.g., a human elongation factor-1α promoter, or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Another aspect of the invention provides an isolated or purified TCR, polypeptide, or protein encoded by any of the nucleic acids or vectors described herein with respect to other aspects of the invention.

Still another aspect of the invention provides an isolated or purified TCR, polypeptide, or protein that results from expression of any of the nucleic acids or vectors described herein with respect to other aspects of the invention.

Another aspect of the invention further provides a host cell comprising any of the nucleic acids or any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. For example, the host cell may be a human lymphocyte. In an aspect of the invention, the host cell is selected from the group consisting of a T cell, a natural killer T (NKT) cell, an invariant natural killer T (iNKT) cell, and a natural killer (NK) cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

Also provided by an aspect of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

In an aspect of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In an aspect, expansion of the numbers of T cells is carried out by culturing the T cells with OKT3 antibody, IL-2, and feeder PBMC (e.g., irradiated allogeneic PBMC).

An aspect of the invention provides a method of producing any of the TCRs, polypeptides, or proteins described herein, the method comprising culturing any of the host cells or populations of host cells described herein, so that the TCR, polypeptide, or protein is produced.

The inventive TCRs, polypeptides, proteins, nucleic acids, recombinant expression vectors, and host cells (including populations thereof), can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, at least about 60%, at least about 70%, at least about 80%, at least about 90%, at least about 95%, or can be about 100%.

The inventive TCRs, polypeptides, proteins, nucleic acids, recombinant expression vectors, and host cells (including populations thereof), all of which are collectively referred to as “inventive TCR materials” hereinafter, can be formulated into a composition, such as a pharmaceutical composition. In this regard, an aspect of the invention provides a pharmaceutical composition comprising any of the TCRs, polypeptides, proteins, nucleic acids, expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agent, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an aspect, the pharmaceutically acceptable carrier is supplemented with human serum albumen.

The amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., mutated p53), or detect, treat or prevent cancer in a period of from about 2 hours or longer, e.g., 12 to 24 or more hours, from the time of administration. In certain aspects, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For example, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In an aspect in which the inventive TCR material is a population of cells, the number of cells administered per infusion may vary, e.g., from about 1×10⁶ to about 1×10¹² cells or more. In certain aspects, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are ideal sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to mutated p53 or to detect, treat, or prevent cancer.

It is contemplated that the inventive pharmaceutical compositions, TCRs, polypeptides, proteins, nucleic acids, recombinant expression vectors, host cells, or populations of cells can be used in methods of treating or preventing cancer. Without being bound to a particular theory, the inventive TCRs are believed to bind specifically to mutated p53, such that the TCR (or related inventive polypeptide or protein), when expressed by a cell, is able to mediate an immune response against a target cell expressing mutated p53. In this regard, an aspect of the invention provides a method of treating or preventing cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

It is also contemplated that the inventive pharmaceutical compositions, TCRs, polypeptides, proteins, nucleic acids, recombinant expression vectors, host cells, or populations of cells can be used in methods of inducing an immune response against a cancer in a mammal. In this regard, an aspect of the invention provides a method of inducing an immune response against a cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to induce an immune response against the cancer in the mammal.

An aspect of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the inducement of an immune response against a cancer in a mammal.

Also provided by an aspect of the invention is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including, e.g., any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colocrectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. In a preferred aspect, the cancer is a cancer which expresses mutated p53. The cancer may express p53 with a mutation at one or more of positions 135, 175, and 237, as defined by SEQ ID NO: 1. The cancer may express p53 with one or more of the following human p53 mutations: C135Y, R175H, or M237I. In an aspect of the invention, the cancer is an epithelial cancer. In an aspect of the invention, the cancer is cholangiocarcinoma, melanoma, colon cancer, rectal cancer, ovarian cancer, endometrial cancer, non-small cell lung cancer (NSCLC), glioblastoma, uterine cervical cancer, head and neck cancer, breast cancer, pancreatic cancer, or bladder cancer. The cancer may be known to comprise a C135Y, R175H, or M237I mutation in human p53.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The following examples further illustrate the invention but, of course, should not be construed as in any way limiting its scope.

### Example 1

This example demonstrates the identification of anti-p53-C135Y reactivity in the TIL of Patient 4316.

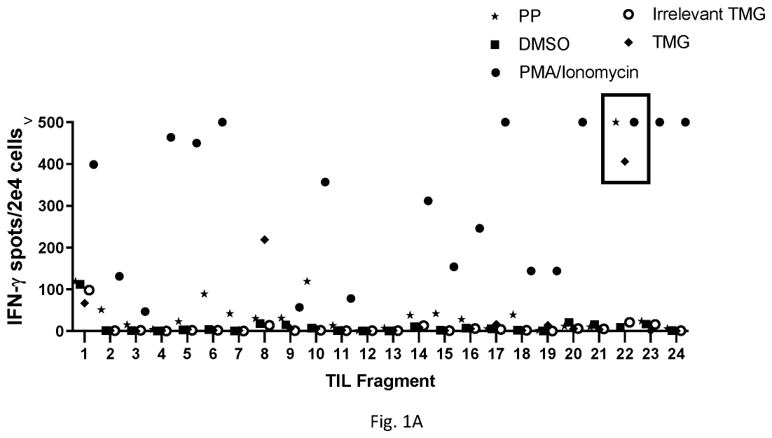

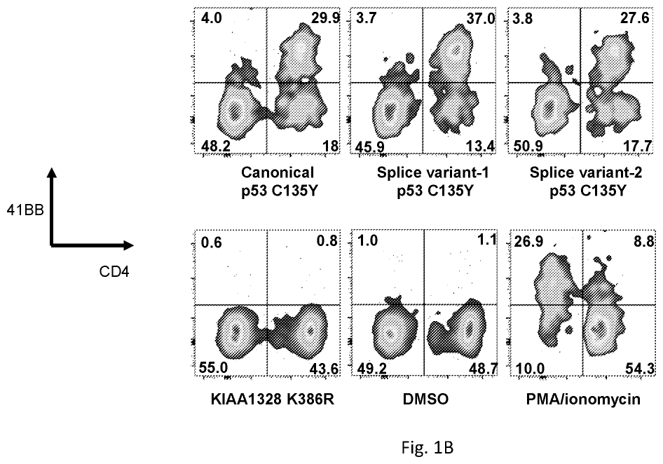

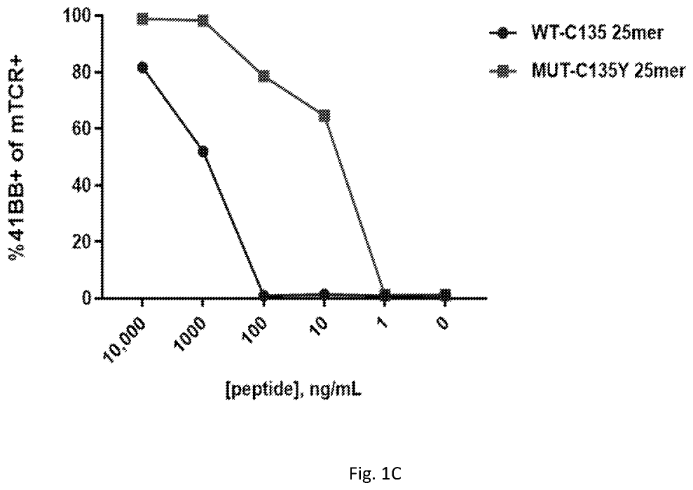

Resected tumors from colorectal cancer Patient 4316 were cut into 24 fragments and were cultured in the presence of the cytokine IL-2 to grow TILs ex vivo. The fragments (numbered F1-F24) were screened against the somatic mutations found in the patient's tumor, including p53 C135Y, by co-culturing the TIL with target cells. The target cells were autologous DCs that were (i) pulsed with PP including p53 C135Y; (ii) transfected with TMG RNA encoding p53 C135Y or irrelevant TMG; or (iii) treated with DMSO (dimethyl sulfoxide) (control). TIL treated with PMA (Phorbol 12-Myristate 13-Acetate)/ionomycin served as a positive control. IFN-γ production was measured by ELISpot assay. The results are shown in FIG. 1A. Fragment number F22 was identified to contain TIL which recognized p53-C135Y.

The TIL from Fragment 22 were parsed and tested for reactivity against p53-C135Y. The target cells were autologous DCs that were pulsed with one of the p53-C135Y peptides shown in Table 1. Target cells pulsed with the irrelevant peptide KIAA1328 K386R or DMSO (vehicle) were included as negative controls. TIL treated with PMA/ionomycin served as a positive control. After co-culture, reactivity was measured by flow cytometric analysis of cell surface 4-1BB expression, a T cell activation marker. The results are shown in FIG. 1B. As shown in FIG. 1B, reactivity was observed following co-culture of TIL with any of the three p53-C135Y peptides shown in Table 1.

TABLE 1

| | | | |
|---|---|---|---|
| TP53-1 | Canonical | TCTYSPALNKMFYQLAKTCPVQLWV | SEQ ID NO: 90 |
| TP53-2 | Splice Variant-1 | NVLYSPALNKMFYQLAKTCPVQLWV | SEQ ID NO: 96 |
| TP53-3 | Splice Variant-2 | SGTAKSVTCTMFYQLAKTCPVQLWV | SEQ ID NO: 97 |

### Example 2

This example demonstrates the isolation of an anti-p53-C135Y TCR from the reactive TIL of Example 1.

Reactive TIL were re-stimulated and sorted by 4-1BB upregulation into 96 well plates for single-cell T-cell receptor (TCR) sequencing. A TCR was found, namely 4316-D TCR.

The sequences of the TCR alpha and beta chain variable regions were identified by single-cell TCR sequencing. The amino acid sequences of the alpha and beta chain variable regions are shown in Table 2. The CDRs are underlined. The N-terminal signal peptides are in bold font.

TABLE 2

| TCR Name | TCR chain | Amino acid sequence |
|---|---|---|
| 4316-D TCR | Variable α | QQVMQIPQYQHVQEGEDFTTYCNSSTTLSNIQWYKQRPGGHP |
| | (Predicted | VFLIQLVKSGEVKKQKRLTFQFGEAKKNSSLHITATQTTDVGT |
| | sequence without | YFCAESYSGGYQKVTFGIGTKLQVIP (SEQ ID NO: 8) |
| | N-terminal signal | |
| | peptide) | |
| | Variable ß | EPEVTQTPSHQVTQMGQEVILRCVPISNHLYFYWYRQILGQKV |
| | (Predicted | EFLVSFYNNEISEKSEIFDDQFSVERPDGSNFTLKIRSTKLEDSA |
| | sequence without | MYFCASSSFSNEQFFGPGTRLTVL (SEQ ID NO: 9) |
| | N-terminal signal | |
| | peptide) | |
| | Variable α | MHLITSMLVLWMQLSQVNGQQVMQIPQYQHVQEGEDFTTY |
| | (With N-terminal | CNSSTTLSNIQWYKQRPGGHPVFLIQLVKSGEVKKQKRLTFQF |
| | signal peptide) | GEAKKNSSLHITATQTTDVGTYFCAESYSGGYQKVTFGIGTKL |
| | | QVIP (SEQ ID NO: 10) |
| | Variable ß | MATWLVCWAIFSLLKAGLTEPEVTQTPSHQVTQMGQEVILR |
| | (With N-terminal | CVPISNHLYFYWYRQILGQKVEFLVSFYNNEISEKSEIFDDQFS |
| | signal peptide) | VERPDGSNFTLKIRSTKLEDSAMYFCASSSFSNEQFFGPGTRLT |
| | | VL (SEQ ID NO: 11) |

### Example 3

This example demonstrates the identification of anti-p53-R175H reactivity in the TIL of Patient 4141.

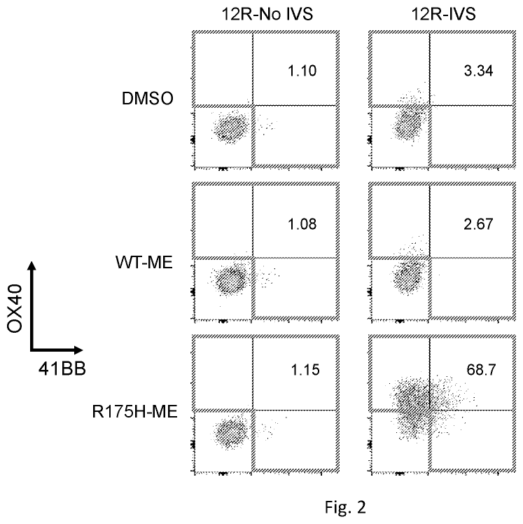

TIL from colorectal cancer patient 4141 were subjected to in vitro sensitization to enrich for neoantigen-reactive T cells, followed by testing against DMSO, the mutant p53 R175H peptide HMTEVVRHC (SEQ ID NO: 92), or the corresponding WT p53 R175 peptide HMTEVVRRC (SEQ ID NO: 91). T cell activation markers, 4-1BB and OX40, were measured by flow cytometry. The results are shown in FIG. 2.

### Example 4

This example demonstrates the isolation of an anti-p53-R175H TCR from the reactive TIL of Example 3.

Reactive TIL were re-stimulated and sorted by 4-1BB upregulation into 96 well plates for single-cell TCR sequencing. A TCR was found, namely 4141 IVS TCR.

The sequences of the TCR alpha and beta chain variable regions were identified by single-cell TCR sequencing. The amino acid sequences of the alpha and beta chain variable regions are shown in Table 3. The CDRs are underlined. The N-terminal signal peptides are in bold font.

TABLE 3

| TCR Name | TCR chain | Amino acid sequence |
|---|---|---|
| 4141 IVS TCR | Variable α | QTVTQSQPEMSVQEAETVTLSCTYDTSENNYYLFWYKQPPSR |
| | (Predicted | QMILVIRQEAYKQQNATENRFSVNFQKAAKSFSLKISDSQLGD |
| | sequence without | TAMYFCAFMAYMEYGNKLVFGAGTILRVKS (SEQ ID NO: 23) |
| | N-terminal signal | |
| | peptide) | |
| | Variable ß | EPEVTQTPSHQVTQMGQEVILRCVPISNHLYFYWYRQILGQKV |
| | (Predicted | EFLVSFYNNEISEKSEIFDDQFSVERPDGSNFTLKIRSTKLEDSA |
| | sequence without | MYFCACKGITDTQYFGPGTRLTVL (SEQ ID NO: 24) |
| | N-terminal signal | |
| | peptide) | |
| | Variable α | MHRVSLLWAVVVSTCLESGMAQTVTQSQPEMSVQEAETVT |
| | (With N-terminal | LSCTYDTSENNYYLFWYKQPPSRQMILVIRQEAYKQQNATEN |
| | signal peptide) | RFSVNFQKAAKSFSLKISDSQLGDTAMYFCAFMAYMEYGNKL |
| | | VFGAGTILRVKS (SEQ ID NO: 25) |
| | Variable ß | MATWLVCWAIFSLLKAGLTEPEVTQTPSHQVTQMGQEVILR |
| | (With N-terminal | CVPISNHLYFYWYRQILGQKVEFLVSFYNNEISEKSEIFDDQFS |
| | signal peptide) | VERPDGSNFTLKIRSTKLEDSAMYFCACKGITDTQYFGPGTRL |
| | | TVL (SEQ ID NO: 26) |

### Example 5

This example demonstrates the identification of anti-p53-M237I reactivity in the TIL of Patient 4304.

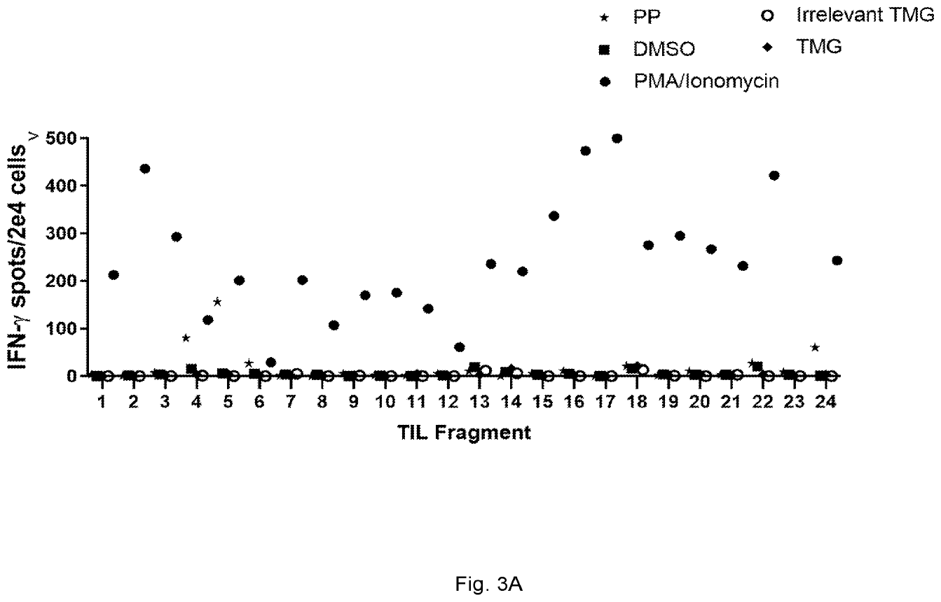

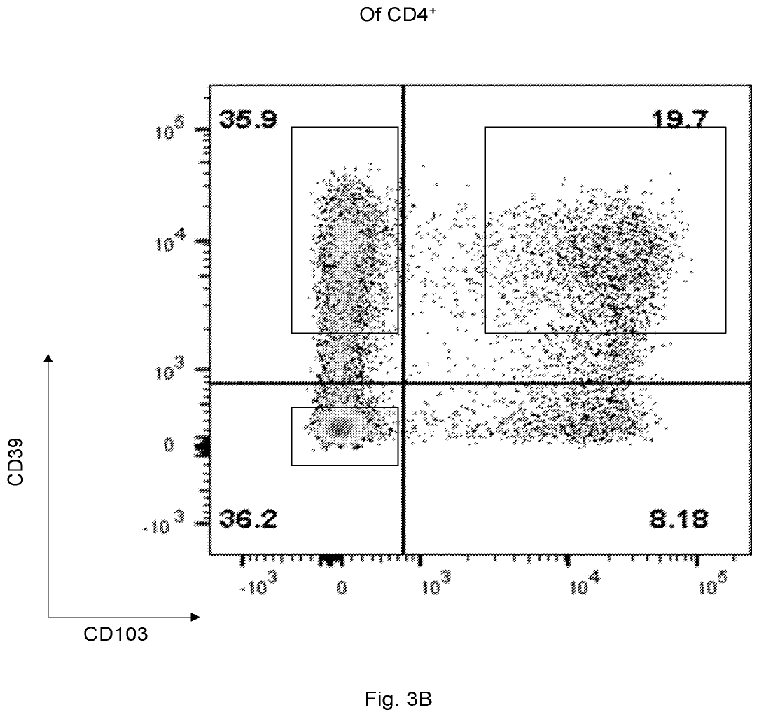

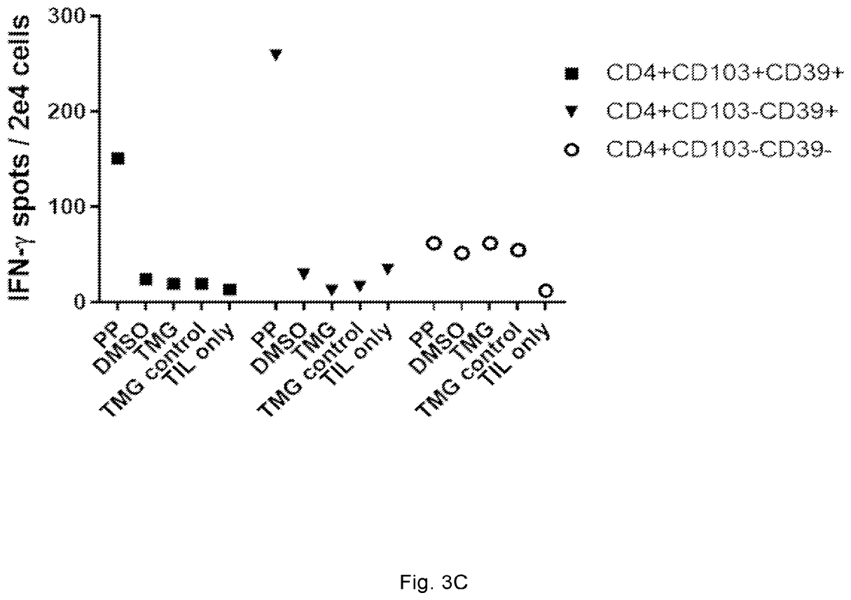

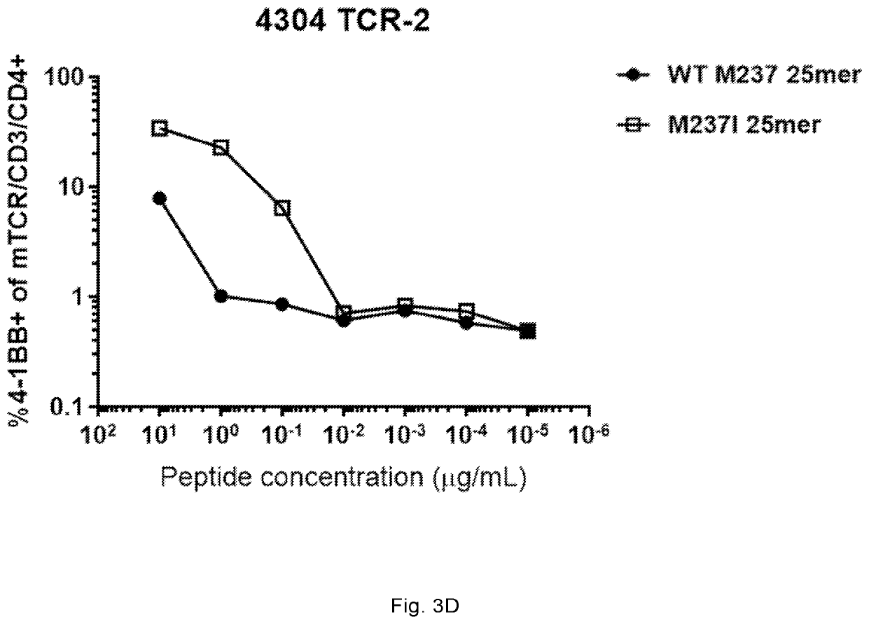

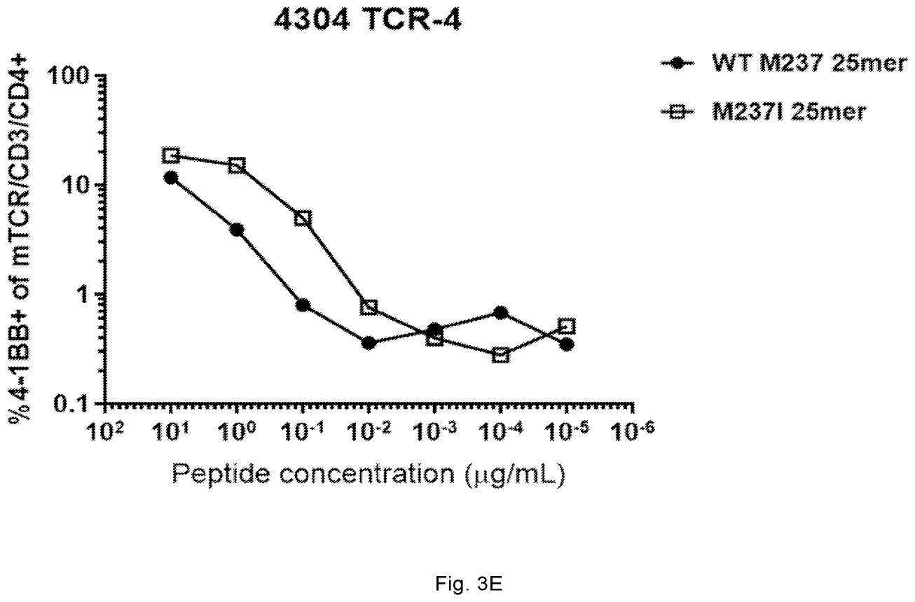

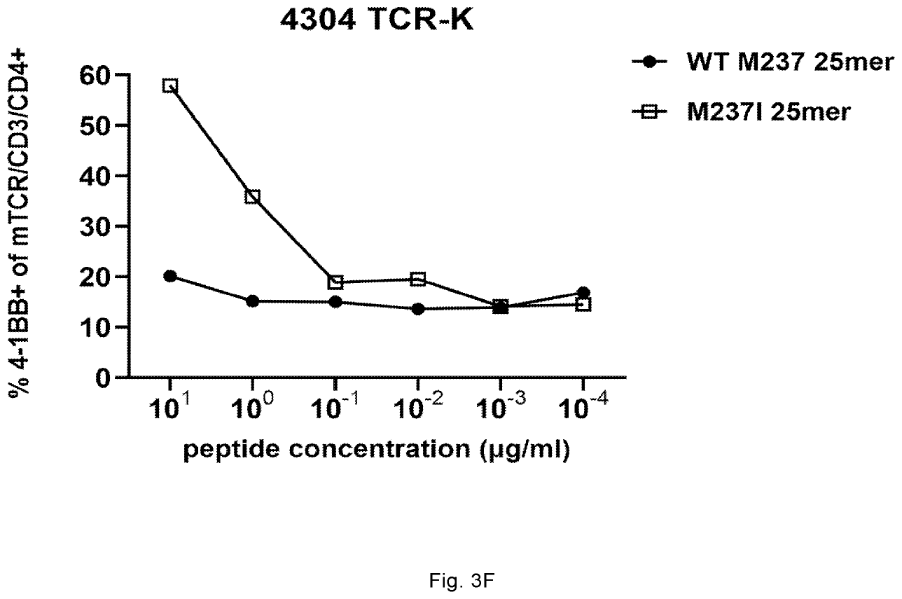

Resected tumors from colorectal cancer Patient 4304 were cut into 24 fragments and were cultured in the presence of the cytokine IL-2 to grow TILs ex vivo. The fragments (numbered F1-F24) were screened against the somatic mutations found in the patient's tumor, including p53 M237I, by co-culturing the TIL with target cells. The target cells were autologous DCs that were (i) pulsed with PP including p53 M237I; (ii) transfected with TMG RNA encoding p53 M237I or irrelevant TMG; or (iii) treated with DMSO (control). TIL treated with PMA/ionomycin served as a positive control. IFN-γ production was measured by ELISpot assay. The results are shown in FIG. 3A. Fragment number F24 was identified to contain TIL which recognized p53-M237I.

The TIL from Fragment 24 were parsed and tested for reactivity against the p53-M237T. The target cells were autologous DCs that were independently pulsed with individual mutant peptides that constituted the peptide pool of FIG. 3A. TIL treated with PMA/ionomycin served as a positive control. Media alone and DCs pulsed with DMSO served as controls. After co-culture, reactivity was tested by measuring IFN-gamma expression by ELISPOT assay. The numbers of IFN-gamma positive spots are shown in Table 4. As shown in Table 4, reactivity was observed following co-culture of TIL with mutated p53 peptide (p53-M237I) VGSDCTTIHYNYICNSSCMGGMNRR (SEQ ID NO: 94). In Table 4, “blank” denotes blank wells without any T cells or target cells.

TABLE 4

| Mutant peptide | Number of IFN-gamma spots |
|---|---|
| ZFN469 | 40 |
| MAP2K2 | ~52 |
| SCN4A | ~46 |
| PPP1R13L | 15 |
| P53 | ~712 |
| PEG3 | 34 |
| DNAH17 | ~86 |
| DMSO | 15 |
| NTN1 | ~33 |
| Media alone | 1 |
| SMAD2 | 23 |
| blank | 2 |
| CPAMD8 | 15 |
| blank | 3 |
| NCAN | 28 |
| PMA/ionomycin | Too numerous to count |

CD4⁺CD103⁺CD39⁺, CD4⁺CD103⁻CD39⁺ or CD4⁺CD103⁻CD39⁻ cells were sorted from tumor digest from patient 4304 by flow cytometry. The gating scheme used for this sorting is shown in FIG. 3B.

The numbers of cells in the sorted populations were expanded by independently co-culturing the sorted cell populations with target cells. The target cells were autologous DCs that were (i) pulsed with PP including p53 M237I; (ii) transfected with TMG RNA encoding p53 M237I or TMG control; or (iii) treated with DMSO (control). The TMG control encoded irrelevant mutations expressed by other patients who did not share the same mutations as patient 4304. TIL cultured alone served as a control. After co-culture, reactivity was tested by measuring IFN-γ secretion by ELISPOT. The results are shown in FIG. 3C. Reactivity was observed following co-culture of sorted CD4⁺CD103⁺CD39⁺ or CD4⁺CD103⁻CD39⁺ cells with target cells pulsed with PP containing p53 M237I.

### Example 6

This example demonstrates the isolation of an anti-p53-M237I TCR from the reactive TIL of Example 5.

Reactive TIL were re-stimulated and sorted by 4-1BB upregulation into 96 well plates for single-cell TCR sequencing. Three TCRs were found, namely 4304 TCR-2, 4304 TCR-4, and 4304 TCR-K.

The sequences of the TCR alpha and beta chain variable regions were identified by single-cell TCR sequencing. The amino acid sequences of the alpha and beta chain variable regions are shown in Table 5. The CDRs are underlined. The N-terminal signal peptides are in bold font.

TABLE 5

| TCR Name | TCR chain | Amino acid sequence |
|---|---|---|
| 4304 TCR-2 | Variable α | QSVSQHNHHVILSEAASLELGCNYSYGGTVNLFWYVQYPGQH |
| | (Predicted | LQLLLKYFSGDPLVKGIKGFEAEFIKSKFSFNLRKPSVQWSDTA |
| | sequence without | EYFCAVTFMDTGRRALTFGSGTRLQVQP (SEQ ID NO: 38) |
| | N-terminal signal | |
| | peptide) | |
| | Variable ß | GVSQSPSNKVTEKGKDVELRCDPISGHTALYWYRQSLGQGLE |
| | (Predicted | FLIYFQGNSAPDKSGLPSDRFSAERTGGSVSTLTIQRTQQEDSA |
| | sequence without | VYLCASSPRGGDYEQYFGPGTRLTVT (SEQ ID NO: 39) |
| | N-terminal signal | |
| | peptide) | |
| | Variable α | MLLLLIPVLGMIFALRDARAQSVSQHNHHVILSEAASLELGC |
| | (With N-terminal | NYSYGGTVNLFWYVQYPGQHLQLLLKYFSGDPLVKGIKGFEA |
| | signal peptide) | EFIKSKFSFNLRKPSVQWSDTAEYFCAVTFMDTGRRALTFGSG |
| | | TRLQVQP (SEQ ID NO: 40) |
| | Variable ß | MATRLLFWVAFCLLGADHTGAGVSQSPSNKVTEKGKDVEL |
| | (With N-terminal | RCDPISGHTALYWYRQSLGQGLEFLIYFQGNSAPDKSGLPSDR |
| | signal peptide) | FSAERTGGSVSTLTIQRTQQEDSAVYLCASSPRGGDYEQYFGP |
| | | GTRLTVT (SEQ ID NO: 41) |
| 4304 TCR-4 | Variable α | QQKEVEQNSGPLSVPEGAIASLNCTYSDRGSQSFFWYRQYSGK |
| | (Predicted | SPELIMFIYSNGDKEDGRFTAQLNKASQYVSLLIRDSQPSDSAT |
| | sequence without | YLCAVRGGNTGFQKLVFGTGTRLLVSP (SEQ ID NO: 53) |
| | N-terminal signal | |
| | peptide) | |
| | Variable ß | AVISQKPSRDICQRGTSLTIQCQVDSQVTMMFWYRQQPGQSLT |
| | (Predicted | LIATANQGSEATYESGFVIDKFPISRPNLTFSTLTVSNMSPEDSSI |
| | sequence without | YLCSVRSEDTQYFGPGTRLTVL (SEQ ID NO: 54) |
| | N-terminal signal | |
| | peptide) | |
| | Variable α | MKSLRVLLVILWLQLSWVWSQQKEVEQNSGPLSVPEGAIAS |
| | (With N-terminal | LNCTYSDRGSQSFFWYRQYSGKSPELIMFIYSNGDKEDGRFTA |
| | signal peptide) | QLNKASQYVSLLIRDSQPSDSATYLCAVRGGNTGFQKLVFGTG |
| | | TRLLVSP (SEQ ID NO: 55) |
| | Variable ß | MASLLLLLLGLGSVFSAVISQKPSRDICQRGTSLTIQCQVDSQ |
| | (With N-terminal | VTMMFWYRQQPGQSLTLIATANQGSEATYESGFVIDKFPISRP |
| | signal peptide) | NLTFSTLTVSNMSPEDSSIYLCSVRSEDTQYFGPGTRLTVL |
| | | (SEQ ID NO: 56) |
| 4304 TCR-K | Variable α | KDQVFQPSTVASSEGAVVEIFCNHSVSNAYNFFWYLHFPGCAP |
| | (Predicted | RLLVKGSKPSQQGRYNMTYERFSSSLLILQVREADAAVYYCA |
| | sequence without | VSGYQLIWGAGTKLIIKPNIQNPEPAV (SEQ ID NO: 68) |
| | N-terminal signal | |
| | peptide) | |
| | Variable ß | GVAQSPRYKIIEKRQSVAFWCNPISGHATLYWYQQILGQGPKL |
| | (Predicted | LIQFQNNGVVDDSQLPKDRFSAERLKGVDSTLKIQPAKLEDSA |
| | sequence without | VYLCASSLDRRGGRETQYFGPGTRLLVL (SEQ ID NO: 69) |
| | N-terminal signal | |
| | peptide) | |
| | Variable α | MHLQSTLGAVWLGLLLNSLWKVAESKDQVFQPSTVASSEG |
| | (With N-terminal | AVVEIFCNHSVSNAYNFFWYLHFPGCAPRLLVKGSKPSQQGR |
| | signal peptide) | YNMTYERFSSSLLILQVREADAAVYYCAVSGYQLIWGAGTKLI |
| | | IKPNIQNPEPAV (SEQ ID NO: 70) |
| | Variable ß | MATRLLCWAALCLLGAELTEAGVAQSPRYKIIEKRQSVAFW |
| | (With N-terminal | CNPISGHATLYWYQQILGQGPKLLIQFQNNGVVDDSQLPKDRF |
| | signal peptide) | SAERLKGVDSTLKIQPAKLEDSAVYLCASSLDRRGGRETQYFG |
| | | PGTRLLVL (SEQ ID NO: 71) |

### Example 7

This example demonstrates the construction of retroviral vectors encoding the respective TCRs of Examples 2, 4, and 6.

Nucleotide sequences encoding the variable regions of the α and β chains of the TCRs of Tables 2, 3, and 5 were obtained and codon optimized. The TCRβ VDJ regions were fused to the mouse TCRβ constant chain. The TCRα VJ regions were fused to the mouse TCRα constant chain. Without being bound to a particular theory or mechanism, it is believed that replacing the constant regions of the human TCRα and TCRβ chains with the corresponding murine constant regions improves TCR expression and functionality (Cohen et al., Cancer Res., 66(17): 8878-86 (2006)).

In addition, the murine TCRα and TCRβ constant chains were cysteine-modified. Transmembrane hydrophobic mutations were introduced into the murine TCRα constant chain. Without being bound to a particular theory or mechanism, it is believed that these modifications result in preferential pairing of the introduced TCR chains and enhanced TCR surface expression and functionality (Cohen et al., Cancer Res., 67(8):3898-903 (2007); Haga-Friedman et al., J. Immu., 188: 5538-5546 (2012)).

To facilitate cloning of the TCR expression cassette into the MSGV1 vector 5′NcoI site, and to introduce a Kozak sequence, the second amino acid in the N-terminal signal peptide of the TCRVα chain was changed to an histidine (H), leucine (L), or lysine (K), and the second amino acid in the N-terminal signal peptide of the TCRVβ chain was changed to an alanine (A).

The full length α and β chains of each of the five TCRs, including these modifications to the constant region, are shown in Table 6. In Table 6, the CDRs are underlined, the constant region is in italics, and the modified amino acid residues of the constant region are underlined and in bold.

TABLE 6

| TCR | | |
|---|---|---|
| Name | TCR chain | Amino acid sequence |
| 4316-D | Cys-substituted, | MHLITSMLVLWMQLSQVNGQQVMQIPQYQHVQEGEDFTTYCNSSTT |
| TCR | LVL-modified α | LSNIQWYKQRPGGHPVFLIQLVKSGEVKKQKRLTFQFGEAKKNSSLHI |
| | chain with N- | TATQTTDVGTYFCAESYSGGYQKVTFGIGTKLQVIPNIQNPEPAVYQLK |
| | terminal signal | DPRSQDSTLCLFTDFDSQINVPKTMESGTFITDKCVLDMKAMDSKSNGAI |
| | peptide | AWSNQTSFTCQDIFKETNATYPSSDVPCDATLTEKSFETDMNLNFQNLLVI |
| | | VLRILLLKVAGFNLLMTLRLWSS (SEQ ID NO: 12) |
| | Cys-substituted, | MATWLVCWAIFSLLKAGLTEPEVTQTPSHQVTQMGQEVILRCVPISN |
| | LVL-modified ß | HLYFYWYRQILGQKVEFLVSFYNNEISEKSEIFDDQFSVERPDGSNFTL |
| | chain with N- | KIRSTKLEDSAMYFCASSSFSNEQFFGPGTRLTVLEDLRNVTPPKVSLFE |
| | terminal signal | PSKAEIANKQKATLVCLARGFFPDHVELSWWVNGKEVHSGVCTDPQAYK |
| | peptide | ESNYSYCLSSRLRVSATFWHNPRNHFRCQVQFHGLSEEDKWPEGSPKPVT |
| | | QNISAEAWGRADCGITSASYQQGVLSATILYEILLGKATLYAVLVSTLVVMA |
| | | MVKRKNS (SEQ ID NO: 13) |
| | Cys-substituted, | QQVMQIPQYQHVQEGEDFTTYCNSSTTLSNIQWYKQRPGGHPVFLIQ |
| | LVL-modified α | LVKSGEVKKQKRLTFQFGEAKKNSSLHITATQTTDVGTYFCAESYSG |
| | chain predicted | GYQKVTFGIGTKLQVIPNIQNPEPAVYQLKDPRSQDSTLCLFTDFDSQIN |
| | sequence without | VPKTMESGTFITDKCVLDMKAMDSKSNGAIAWSNQTSFTCQDIFKETNAT |
| | N-terminal | YPSSDVPCDATLTEKSFETDMNLNFQNLLVIVLRILLLKVAGFNLLMTLRL |
| | signal peptide | WSS (SEQ ID NO: 14) |
| | Cys-substituted, | EPEVTQTPSHQVTQMGQEVILRCVPISNHLYFYWYRQILGQKVEFLVS |
| | LVL-modified ß | FYNNEISEKSEIFDDQFSVERPDGSNFTLKIRSTKLEDSAMYFCASSSFS |
| | chain predicted | NEQFFGPGTRLTVLEDLRNVTPPKVSLFEPSKAEIANKQKATLVCLARGF |
| | sequence without | FPDHVELSWWVNGKEVHSGVCTDPQAYKESNYSYCLSSRLRVSATFWHNP |
| | N-terminal | RNHFRCQVQFHGLSEEDKWPEGSPKPVTQNISAEAWGRADCGITSASYQ |
| | signal peptide | QGVLSATILYEILLGKATLYAVLVSTLVVMAMVKRKNS (SEQ ID NO: 15) |
| 4141 | Cys-substituted, | MHRVSLLWAVVVSTCLESGMAQTVTQSQPEMSVQEAETVTLSCTYD |
| IVS | LVL-modified α | TSENNYYLFWYKQPPSRQMILVIRQEAYKQQNATENRFSVNFQKAAK |
| TCR | chain with N- | SFSLKISDSQLGDTAMYFCAFMAYMEYGNKLVFGAGTILRVKSNIQNP |
| | terminal signal | EPAVYQLKDPRSQDSTLCLFTDFDSQINVPKTMESGTFITDKCVLDMKAM |
| | peptide | DSKSNGAIAWSNQTSFTCQDIFKETNATYPSSDVPCDATLTEKSFETDMNL |
| | | NFQNLLVIVLRILLLKVAGFNLLMTLRLWSS (SEQ ID NO: 27) |
| | Cys-substituted, | MATWLVCWAIFSLLKAGLTEPEVTQTPSHQVTQMGQEVILRCVPISN |
| | LVL-modified ß | HLYFYWYRQILGQKVEFLVSFYNNEISEKSEIFDDQFSVERPDGSNFTL |
| | chain with N- | KIRSTKLEDSAMYFCACKGITDTQYFGPGTRLTVLEDLRNVTPPKVSLF |
| | terminal signal | EPSKAELANKQKATLVCLARGFFPDHVELSWWVNGKEVHSGVCTDPQAY |
| | peptide | KESNYSYCLSSRLRVSATFWHNPRNHFRCQVQFHGLSEEDKWPEGSPKPV |
| | | TQNISAEAWGRADCGITSASYQQGVLSATILYEILLGKATLYAVLVSTLVVMA |
| | | MVKRKNS (SEQ ID NO: 28) |
| | Cys-substituted, | QTVTQSQPEMSVQEAETVTLSCTYDTSENNYYLFWYKQPPSRQMILVI |
| | LVL-modified α | RQEAYKQQNATENRFSVNFQKAAKSFSLKISDSQLGDTAMYFCAFMA |
| | chain predicted | YMEYGNKLVFGAGTILRVKSNIQNPEPAVYQLKDPRSQDSTLCLFTDFD |
| | sequence without | SQINVPKTMESGTFITDKCVLDMKAMDSKSNGAIAWSNQTSFTCQDIFKE |
| | N-terminal | TNATYPSSDVPCDATLTEKSFETDMNLNFQNLLVIVLRILLLKVAGENLLM |
| | signal peptide | TLRLWSS (SEQ ID NO: 29) |
| | Cys-substituted, | EPEVTQTPSHQVTQMGQEVILRCVPISNHLYFYWYRQILGQKVEFLVS |
| | LVL-modified ß | FYNNEISEKSEIFDDQFSVERPDGSNFTLKIRSTKLEDSAMYFCACKGIT |
| | chain predicted | DTQYFGPGTRLTVLEDLRNVTPPKVSLFEPSKAELANKQKATLVCLARGF |
| | sequence without | FPDHVELSWWVNGKEVHSGVCTDPQAYKESNYSYCLSSRLRVSATFWHNP |
| | N-terminal | RNHFRCQVQFHGLSEEDKWPEGSPKPVTQNISAEAWGRADCGITSASYQ |
| | signal peptide | QGVLSATILYEILLGKATLYAVLVSTLVVMAMVKRKNS (SEQ ID NO: 30) |
| 4304 | Cys-substituted, | MLLLLIPVLGMIFALRDARAQSVSQHNHHVILSEAASLELGCNYSYGG |
| TCR-2 | LVL-modified α | TVNLFWYVQYPGQHLQLLLKYFSGDPLVKGIKGFEAEFIKSKFSFNLR |
| | chain with N- | KPSVQWSDTAEYFCAVTFMDTGRRALTFGSGTRLQVQPNIQNPEPAVY |
| | terminal signal | QLKDPRSQDSTLCLFTDFDSQINVPKTMESGTFITDKCVLDMKAMDSKSN |
| | peptide | GAIAWSNQTSFTCQDIFKETNATYPSSDVPCDATLTEKSFETDMNLNFQNL |
| | | LVIVLRILLLKVAGFNLLMTLRLWSS (SEQ ID NO: 42) |
| | Cys-substituted, | MATRLLFWVAFCLLGADHTGAGVSQSPSNKVTEKGKDVELRCDPISG |
| | LVL-modified ß | HTALYWYRQSLGQGLEFLIYFQGNSAPDKSGLPSDRFSAERTGGSVST |
| | chain with N- | LTIQRTQQEDSAVYLCASSPRGGDYEQYFGPGTRLTVTEDLRNVTPPK |
| | terminal signal | VSLFEPSKAELANKQKATLVCLARGFFPDHVELSWWVNGKEVHSGVCTDP |
| | peptide | QAYKESNYSYCLSSRLRVSATFWHNPRNHFRCQVQFHGLSEEDKWPEGSP |
| | | KPVTQNISAEAWGRADCGITSASYQQGVLSATILYEILLGKATLYAVLVSTLV |
| | | VMAMVKRKNS (SEQ ID NO: 43) |
| | Cys-substituted, | QSVSQHNHHVILSEAASLELGCNYSYGGTVNLFWYVQYPGQHLQLLL |
| | LVL-modified α | KYFSGDPLVKGIKGFEAEFIKSKFSFNLRKPSVQWSDTAEYFCAVTFM |
| | chain predicted | DTGRRALTFGSGTRLQVQPNIQNPEPAVYQLKDPRSQDSTLCLFTDFDS |
| | sequence without | QINVPKTMESGTFITDKCVLDMKAMDSKSNGAIAWSNQTSFTCQDIFKET |
| | N-terminal | NATYPSSDVPCDATLTEKSFETDMNLNFQNLLVIVLRILLLKVAGFNLLMT |
| | signal peptide | LRLWSS (SEQ ID NO: 44) |
| | Cys-substituted, | GVSQSPSNKVTEKGKDVELRCDPISGHTALYWYRQSLGQGLEFLIYFQ |
| | LVL-modified ß | GNSAPDKSGLPSDRFSAERTGGSVSTLTIQRTQQEDSAVYLCASSPRG |
| | chain predicted | GDYEQYFGPGTRLTVTEDLRNVTPPKVSLFEPSKAELANKQKATLVCLAR |
| | sequence without | GFFPDHVELSWWVNGKEVHSGVCTDPQAYKESNYSYCLSSRLRVSATFWH |
| | N-terminal | NPRNHFRCQVQFHGLSEEDKWPEGSPKPVTQNISAEAWGRADCGITSASY |
| | signal peptide | QQGVLSATILYEILLGKATLYAVLVSTLVVMAMVKRKNS (SEQ ID NO: 45) |
| 4304 | Cys-substituted, | MKSLRVLLVILWLQLSWVWSQQKEVEQNSGPLSVPEGAIASLNCTYS |
| TCR-4 | LVL-modified α | DRGSQSFFWYRQYSGKSPELIMFIYSNGDKEDGRFTAQLNKASQYVS |
| | chain with N- | LLIRDSQPSDSATYLCAVRGGNTGFQKLVFGTGTRLLVSPNIQNPEPAV |
| | terminal signal | YQLKDPRSQDSTLCLFTDFDSQINVPKTMESGTFITDKCVLDMKAMDSKS |
| | peptide | NGALAWSNQTSFTCQDIFKETNATYPSSDVPCDATLTEKSFETDMNLNFQN |
| | | LLVIVLRILLLKVAGFNLLMTLRLWSS (SEQ ID NO: 57) |
| | Cys-substituted, | MASLLLLLLGLGSVFSAVISQKPSRDICQRGTSLTIQCQVDSQVTMMF |
| | LVL-modified ß | WYRQQPGQSLTLIATANQGSEATYESGFVIDKFPISRPNLTFSTLTVSN |
| | chain with N- | MSPEDSSIYLCSVRSEDTQYFGPGTRLTVLEDLRNVTPPKVSLFEPSKAE |
| | terminal signal | IANKQKATLVCLARGFFPDHVELSWWVNGKEVHSGVCTDPQAYKESNYSY |
| | peptide | CLSSRLRVSATFWHNPRNHFRCQVQFHGLSEEDKWPEGSPKPVTQNISAE |
| | | AWGRADCGITSASYQQGVLSATILYEILLGKATLYAVLVSTLVVMAMVKRK |
| | | NS (SEQ ID NO: 58) |
| | Cys-substituted, | QQKEVEQNSGPLSVPEGAIASLNCTYSDRGSQSFFWYRQYSGKSPELI |
| | LVL-modified α | MFIYSNGDKEDGRFTAQLNKASQYVSLLIRDSQPSDSATYLCAVRGG |
| | chain predicted | NTGFQKLVFGTGTRLLVSPNIQNPEPAVYQLKDPRSQDSTLCLFTDFDS |
| | sequence without | QINVPKTMESGTFITDKCVLDMKAMDSKSNGAIAWSNQTSFTCQDIFKET |
| | N-terminal | NATYPSSDVPCDATLTEKSFETDMNLNFQNLLVIVLRILLLKVAGFNLLMT |
| | signal peptide | LRLWSS (SEQ ID NO: 59) |
| | Cys-substituted, | AVISQKPSRDICQRGTSLTIQCQVDSQVTMMFWYRQQPGQSLTLIATA |
| | LVL-modified ß | NQGSEATYESGFVIDKFPISRPNLTFSTLTVSNMSPEDSSIYLCSVRSED |
| | chain predicted | TQYFGPGTRLTVLEDLRNVTPPKVSLFEPSKAELANKQKATLVCLARGFF |
| | sequence without | PDHVELSWWVNGKEVHSGVCTDPQAYKESNYSYCLSSRLRVSATFWHNPR |
| | N-terminal | NHFRCQVQFHGLSEEDKWPEGSPKPVTQNISAEAWGRADCGITSASYQQ |
| | signal peptide | GVLSATILYEILLGKATLYAVLVSTLVVMAMVKRKNS (SEQ ID NO: 60) |
| 4304 | Cys-substituted, | MHLQSTLGAVWLGLLLNSLWKVAESKDQVFQPSTVASSEGAVVEIFC |
| TCR-K | LVL-modified α | NHSVSNAYNFFWYLHFPGCAPRLLVKGSKPSQQGRYNMTYERFSSSL |
| | chain with N- | LILQVREADAAVYYCAVSGYQLIWGAGTKLIIKPNIQNPEPAVNIQNPE |
| | terminal signal | PAVYQLKDPRSQDSTLCLFTDFDSQINVPKTMESGTFITDKCVLDMKAMD |
| | peptide | SKSNGAIAWSNQTSFTCQDIFKETNATYPSSDVPCDATLTEKSFETDMNLN |
| | | FQNLLVIVLRILLLKVAGFNLLMTLRLWSS (SEQ ID NO: 72) |
| | Cys-substituted, | MATRLLCWAALCLLGAELTEAGVAQSPRYKIIEKRQSVAFWCNPISG |
| | LVL-modified ß | HATLYWYQQILGQGPKLLIQFQNNGVVDDSQLPKDRFSAERLKGVDS |
| | chain with N- | TLKIQPAKLEDSAVYLCASSLDRRGGRETQYFGPGTRLLVLEDLRNVT |
| | terminal signal | PPKVSLFEPSKAELANKQKATLVCLARGFFPDHVELSWWVNGKEVHSGVC |
| | peptide | TDPQAYKESNYSYCLSSRLRVSATFWHNPRNHFRCQVQFHGLSEEDKWPE |
| | | GSPKPVTQNISAEAWGRADCGITSASYQQGVLSATILYEILLGKATLYAVLVS |
| | | TLVVMAMVKRKNS (SEQ ID NO: 73) |
| | Cys-substituted, | KDQVFQPSTVASSEGAVVEIFCNHSVSNAYNFFWYLHFPGCAPRLLV |
| | LVL-modified α | KGSKPSQQGRYNMTYERFSSSLLILQVREADAAVYYCAVSGYQLIWG |
| | chain predicted | AGTKLIIKPNIQNPEPAVNIQNPEPAVYQLKDPRSQDSTLCLFTDFDSQIN |
| | sequence without | VPKTMESGTFITDKCVLDMKAMDSKSNGAIAWSNQTSFTCQDIFKETNAT |
| | N-terminal | YPSSDVPCDATLTEKSFETDMNLNFQNLLVIVLRILLLKVAGENLLMTLRL |
| | signal peptide | WSS (SEQ ID NO: 74) |
| | Cys-substituted, | GVAQSPRYKIIEKRQSVAFWCNPISGHATLYWYQQILGQGPKLLIQFQ |
| | LVL-modified ß | NNGVVDDSQLPKDRFSAERLKGVDSTLKIQPAKLEDSAVYLCASSLD |
| | chain predicted | RRGGRETQYFGPGTRLLVLEDLRNVTPPKVSLFEPSKAELANKQKATLVC |
| | sequence without | LARGFFPDHVELSWWVNGKEVHSGVCTDPQAYKESNYSYCLSSRLRVSAT |
| | N-terminal | FWHNPRNHFRCQVQFHGLSEEDKWPEGSPKPVTQNISAEAWGRADCGIT |
| | signal peptide | SASYQQGVLSATILYEILLGKATLYAVLVSTLVVMAMVKRKNS (SEQ ID NO: |
| | | 75) |

Nucleotide sequences encoding the α and β chains of the TCRs of Table 6 were cloned into an MSGV1-based retroviral vector with the following expression cassette configuration: 5′ NcoI-VDJβ-mCj3-Furin/SerGly/P2A-VJα-mCα-EcoRI3′.

The TCRβ and TCRα chains were separated by a Furin Ser/Gly P2A linker RAKRSGSGATNFSLLKQAGDVEENPGP (SEQ ID NO: 80). Without being bound to a particular theory or mechanism, it is believed that the linker provides comparable expression efficiency of the two chains (Szymczak et al., Nat. Biotechnol., 22(5):589-94 (2004)).

The TCR expression cassette of the retroviral vector encoded, from 5′ to 3′, the TCRβ and TCRα chains separated by the linker. The amino acid sequences encoded by each respective TCR expression cassette is shown in Table 7. In Table 7, the CDRs are underlined, the constant regions are italicized, and the linker is shown in bold.

TABLE 7

| TCR Name | Amino acid sequence encoded by TCR Expression Cassette |
|---|---|
| 4316-D TCR | MATWLVCWAIFSLLKAGLTEPEVTQTPSHQVTQMGQEVILRCVPISNHLYFYWY |
| | RQILGQKVEFLVSFYNNEISEKSEIFDDQFSVERPDGSNFTLKIRSTKLEDSAMYFC |
| | ASSSFSNEQFFGPGTRLTVLEDLRNVTPPKVSLFEPSKAELANKQKATLVCLARGFFP |
| | DHVELSWWVNGKEVHSGVCTDPQAYKESNYSYCLSSRLRVSATFWHNPRNHFRCQVQ |
| | FHGLSEEDKWPEGSPKPVTQNISAEAWGRADCGITSASYQQGVLSATILYEILLGKATL |
| | YAVLVSTLVVMAMVKRKNSRAKRSGSGATNFSLLKQAGDVEENPGPMHLITSML |
| | VLWMQLSQVNGQQVMQIPQYQHVQEGEDFTTYCNSSTTLSNIQWYKQRPGGHP |
| | VFLIQLVKSGEVKKQKRLTFQFGEAKKNSSLHITATQTTDVGTYFCAESYSGGYQ |
| | KVTFGIGTKLQVIPNIQNPEPAVYQLKDPRSQDSTLCLFTDFDSQINVPKTMESGTFIT |
| | DKCVLDMKAMDSKSNGAIAWSNQTSFTCQDIFKETNATYPSSDVPCDATLTEKSFETD |
| | MNLNFQNLLVIVLRILLLKVAGFNLLMTLRLWSS |
| | (SEQ ID NO: 16) |
| 4141 IVS TCR | MATWLVCWAIFSLLKAGLTEPEVTQTPSHQVTQMGQEVILRCVPISNHLYFYWY |
| | RQILGQKVEFLVSFYNNEISEKSEIFDDQFSVERPDGSNFTLKIRSTKLEDSAMYFC |
| | ACKGITDTQYFGPGTRLTVLEDLRNVTPPKVSLFEPSKAELANKQKATLVCLARGFF |
| | PDHVELSWWVNGKEVHSGVCTDPQAYKESNYSYCLSSRLRVSATFWHNPRNHFRCQV |
| | QFHGLSEEDKWPEGSPKPVTQNISAEAWGRADCGITSASYQQGVLSATILYEILLGKAT |
| | LYAVLVSTLVVMAMVKRKNSRAKRSGSGATNFSLLKQAGDVEENPGPMHRVSLL |
| | WAVVVSTCLESGMAQTVTQSQPEMSVQEAETVTLSCTYDTSENNYYLFWYKQP |
| | PSRQMILVIRQEAYKQQNATENRFSVNFQKAAKSFSLKISDSQLGDTAMYFCAF |
| | MAYMEYGNKLVFGAGTILRVKSNIQNPEPAVYQLKDPRSQDSTLCLFTDFDSQINV |
| | PKTMESGTFITDKCVLDMKAMDSKSNGAIAWSNQTSFTCQDIFKETNATYPSSDVPCD |
| | ATLTEKSFETDMNLNFQNLLVIVLRILLLKVAGFNLLMTLRLWSS |
| | (SEQ ID NO: 31) |
| 4304 TCR-2 | MATRLLFWVAFCLLGADHTGAGVSQSPSNKVTEKGKDVELRCDPISGHTALYW |
| | YRQSLGQGLEFLIYFQGNSAPDKSGLPSDRFSAERTGGSVSTLTIQRTQQEDSAVY |
| | LCASSPRGGDYEQYFGPGTRLTVTEDLRNVTPPKVSLFEPSKAELANKQKATLVCLA |
| | RGFFPDHVELSWWVNGKEVHSGVCTDPQAYKESNYSYCLSSRLRVSATFWHNPRNHF |
| | RCQVQFHGLSEEDKWPEGSPKPVTQNISAEAWGRADCGITSASYQQGVLSATILYEILL |
| | GKATLYAVLVSTLVVMAMVKRKNSRAKRSGSGATNFSLLKQAGDVEENPGPMLL |
| | LLIPVLGMIFALRDARAQSVSQHNHHVILSEAASLELGCNYSYGGTVNLFWYVQ |
| | YPGQHLQLLLKYFSGDPLVKGIKGFEAEFIKSKFSFNLRKPSVQWSDTAEYFCAV |
| | TFMDTGRRALTFGSGTRLQVQPNIQNPEPAVYQLKDPRSQDSTLCLFTDFDSQINVP |
| | KTMESGTFITDKCVLDMKAMDSKSNGAIAWSNQTSFTCQDIFKETNATYPSSDVPCDA |
| | TLTEKSFETDMNLNFQNLLVIVLRILLLKVAGFNLLMTLRLWSS |
| | (SEQ ID NO: 46) |
| 4304 TCR-4 | MASLLLLLLGLGSVFSAVISQKPSRDICQRGTSLTIQCQVDSQVTMMFWYRQQPG |
| | QSLTLIATANQGSEATYESGFVIDKFPISRPNLTFSTLTVSNMSPEDSSIYLCSVRSE |
| | DTQYFGPGTRLTVLEDLRNVTPPKVSLFEPSKAEIANKQKATLVCLARGFFPDHVELS |
| | WWVNGKEVHSGVCTDPQAYKESNYSYCLSSRLRVSATFWHNPRNHFRCQVQFHGLSE |
| | EDKWPEGSPKPVTQNISAEAWGRADCGITSASYQQGVLSATILYEILLGKATLYAVLVST |
| | LVVMAMVKRKNSRAKRSGSGATNFSLLKQAGDVEENPGPMKSLRVLLVILWLQ |
| | LSWVWSQQKEVEQNSGPLSVPEGAIASLNCTYSDRGSQSFFWYRQYSGKSPELI |
| | MFIYSNGDKEDGRFTAQLNKASQYVSLLIRDSQPSDSATYLCAVRGGNTGFQKL |
| | VFGTGTRLLVSPNIQNPEPAVYQLKDPRSQDSTLCLFTDFDSQINVPKTMESGTFITD |
| | KCVLDMKAMDSKSNGAIAWSNQTSFTCQDIFKETNATYPSSDVPCDATLTEKSFETD |
| | MNLNFQNLLVIVLRILLLKVAGFNLLMTLRLWSS |
| | (SEQ ID NO: 61) |
| 4304 TCR-K | MATRLLCWAALCLLGAELTEAGVAQSPRYKIIEKRQSVAFWCNPISGHATLYWY |
| | QQILGQGPKLLIQFQNNGVVDDSQLPKDRFSAERLKGVDSTLKIQPAKLEDSAVY |
| | LCASSLDRRGGRETQYFGPGTRLLVLEDLRNVTPPKVSLFEPSKAELANKQKATLVC |
| | LARGFFPDHVELSWWVNGKEVHSGVCTDPQAYKESNYSYCLSSRLRVSATFWHNPRN |
| | HFRCQVQFHGLSEEDKWPEGSPKPVTQNISAEAWGRADCGITSASYQQGVLSATILYE |
| | ILLGKATLYAVLVSTLVVMAMVKRKNSRAKRSGSGATNFSLLKQAGDVEENPGPM |
| | HLQSTLGAVWLGLLLNSLWKVAESKDQVFQPSTVASSEGAVVEIFCNHSVSNAY |
| | NFFWYLHFPGCAPRLLVKGSKPSQQGRYNMTYERFSSSLLILQVREADAAVYYC |
| | AVSGYQLIWGAGTKLIIKPNIQNPEPAVNIQNPEPAVYQLKDPRSQDSTLCLFTDFD |
| | SQINVPKTMESGTFITDKCVLDMKAMDSKSNGAIAWSNQTSFTCQDIFKETNATYPSS |
| | DVPCDATLTEKSFETDMNLNFQNLLVIVLRILLLKVAGFNLLMTLRLWSS |
| | (SEQ ID NO: 76) |

### Example 8

This example demonstrates the avidity of the 4316-D TCR encoded by the retroviral vector of Example 7.

Healthy donor peripheral blood lymphocytes (PBLs) were transduced with the 4316-D TCR retroviral vector of Example 7 (effector cells). Target cells were autologous immature DCs pulsed with serially diluted 25-mer peptides p53-C135Y TCTYSPALNKMFYQLAKTCPVQLWV (SEQ ID NO: 90) or WT p53-C135 TCTYSPALNKMFCQLAKTCPVQLWV (SEQ ID NO: 89).

The avidity of CD4 4316-D TCR was determined by co-culturing effector cells with the target cells. Reactivity was measured by determining the percentage of murine TCR constant region-expressing T cells expressing 4-1BB. The results are shown in FIG. 1C. The transduced cells recognized p53-C135Y.

### Example 9

This example demonstrates that the 4316-D TCR encoded by the retroviral vector of Example 7 recognizes p53-C135Y presented by an HLA-DRB1*07:01/HLA-DRA1*01:01 heterodimer.

The MHC Class II molecules expressed by Patient 4316 were determined using exome and mRNA sequencing. The expressed MHC Class II molecules are shown in Table 8.

TABLE 8
MHC Class II molecules expressed by Patient 4316

DPA1*01:03-DPB1*02:01
DPA1*01:03-DPB1*11:01
DPA1*02:01-DPB1*02:01
DPA1*02:01-DPB1*11:01
DQA1*01:01-DQB1*02:01
DQA1*01:01-DQB1*02:02
DQA1*01:01-DQB1*05:01
DQA1*02:01-DQB1*02:01
DQA1*02:01-DQB1*02:02
DQA1*02:01-DQB1*05:01
DRA*01:01-DRB1*07:01
DRA*01:01-DRB3*01:01
DQA*01:01-DRB4*01:01

Effector cells were allogeneic PBL transduced with the 4316-D TCR retroviral vector of Example 7. Target cells were 30,000 COS7 cells independently transfected with one of the HLA Class II heterodimers shown in Table 8 and pulsed with DMSO, wild-type p53-C135 25-mer peptide TCTYSPALNKMFCQLAKTCPVQLWV (SEQ ID NO: 89) (1 pg/mL), or p53-C135Y 25-mer peptide TCTYSPALNKMFYQLAKTCPVQLWV (SEQ ID NO: 90) (1 pg/mL).

After co-culture of 20,000 effector cells with target cells for 18 hours, reactivity was tested by measuring IFN-gamma expression by ELISPOT assay. Reactivity was observed only upon co-culture of the 4316-D TCR-transduced cells with the p53-C135Y 25-mer-loaded target cells which had been transduced with a nucleotide sequence encoding an HLA-DRA*01:01/HLA-DRB1*07:01 heterodimer. These data show that the 4316-D TCR is restricted by DRB1*07:01.

### Example 10

This example demonstrates the avidity and specificity of the 4141 IVS TCR encoded by the retroviral vector of Example 7.

Effector cells were healthy donor PBL transduced with the 4141 IVS TCR retroviral vector of Example 7. Target cells were HLA-A*02⁺ T2 leukemia cells pulsed with serially diluted ME p53-R175H peptide HMTEVVRHC (SEQ ID NO: 92) or WT p53-R175 peptide HMTEVVRRC (SEQ ID NO: 91) at the concentrations shown in Table 9.

TABLE 9
Concentration of pulsed peptide

| | |
|---|---|
| 10 | μg/mL |
| 1 | μg/mL |
| 100 | ng/mL |
| 10 | ng/mL |
| 1 | ng/mL |
| 100 | pg/mL |
| 10 | pg/mL |

The avidity and specificity of 4141 IVS TCR was determined by co-culturing 20,000 effector cells with 100,000 target cells for 18 hours. IFN-gamma production was measured by ELISPOT assay (N=3). The results showed that the cells transduced with the 4141 IVS TCR recognized the ME p53-R175H peptide pulsed at concentrations of 100 pg/mL or higher. The cells transduced with the 4141 IVS TCR did not recognize the WT p53-R175 peptide. These data show that the 4141 IVS TCR is highly specific for mutant p53 R175H.

### Example 11

This example demonstrates that the 4141 IVS TCR encoded by the retroviral vector of Example 7 recognizes p53-R175H presented by HLA-A*02:01.

The MHC Class I molecules expressed by Patient 4141 were determined using exome and mRNA sequencing. The expressed MHC Class I molecules are shown in Table 10.

TABLE 10
MHC Class I molecules expressed by Patient 4141

A02:01
A23:01
B08:01
B44:03
C04:01
C07:01
HLA-ALL

Effector cells were healthy donor PBLs transduced with the 4141 IVS TCR retroviral vector of Example 7. Target cells were 30,000 COS7 cells independently transfected with one of the HLA Class I molecules shown in Table 10 and pulsed with DMSO, ME p53-R175H peptide HMTEVVRHC (SEQ ID NO: 92) (1 pg/mL) or WT p53-R175 peptide HMTEVVRRC (SEQ ID NO: 91) (1 pg/mL). HLA-ALL in Table 10 refers to target cells that expressed all six of the HLA Class I molecules shown in Table 10, which served as a positive control.

After co-culture of 20,000 effector cells with target cells for 18 hours, reactivity was tested by measuring IFN-gamma expression by ELISPOT assay. Reactivity was observed only upon co-culture of the 4141 IVS TCR-transduced cells with the p53-R175H 9-mer-loaded target cells which had been transduced with a nucleotide sequence encoding HLA-A*02:01. These data show that the 4141 IVS TCR is restricted by HLA-A*02:01.

### Example 12

This example demonstrates the avidity of the 4304 TCR-4, 4304 TCR-K, or 4304 TCR-2 encoded by the respective retroviral vectors of Example 7.

Healthy donor PBLs were independently transduced with the 4304 TCR-4, 4304 TCR-K, or 4304 TCR-2 retroviral vector of Example 7 (effector cells). Target cells were autologous immature DCs pulsed with serially diluted 25-mer peptide p53-M237I VGSDCTTIHYNYICNSSCMGGMNRR (SEQ ID NO: 94) or WT p53-M237 VGSDCTTIHYNYMCNSSCMGGMNRR (SEQ ID NO: 93).

The avidities of the TCRs were determined by co-culturing effector cells with the target cells. Reactivity was measured by determining the percentage of murine TCR constant region-expressing, CD3⁺CD4⁺ T cells expressing 4-1BB. The results are shown in FIGS. 3D-F. The transduced cells recognized p53-M237I.

### Example 13

This example demonstrates that the 4304 TCR-4, 4304 TCR-K, or 4304 TCR-2 encoded by the respective retroviral vectors of Example 7 recognize p53-M237I presented by an HLA-DRB1*01:01/HLA-DRA1*01:01 heterodimer.

The MHC Class II molecules expressed by Patient 4304 were determined using exome and mRNA sequencing. The expressed MHC Class II molecules are shown in Table 11.

TABLE 11
MHC Class II molecules expressed by Patient 4304

| | |
|---|---|
| DRA1*01:01 | DQA1*02:01 |
| DRB1*01:01 | DQB1*02:01 |
| DRA1*01:01 | DQA1*02:01 |
| DRB1*07:01 | DQB1*05:01 |
| DRA1*01:01 | DPA1*01:03 |
| DRB3*01:01 | DPB1*04:02 |
| DRA1*01:01 | DPA1*01:03 |
| DRB4*01:01 | DPB1*11:01 |
| DQA1*01:01 | DPA1*02:01 |
| DQB1*02:01 | DPB1*04:02 |
| DQA1*01:01 | DPA1*02:01 |
| DQB1*05:01 | DPB1*11:01 |

Effector cells were healthy donor PBLs transduced with the 4304 TCR-4, 4304 TCR-K, or 4304 TCR-2 encoded by the respective retroviral vectors of Example 7. Target cells were 30,000 COS7 cells independently transfected with one of the HLA Class II heterodimers shown in Table 11 and pulsed with DMSO, p53-M237I peptide VGSDCTTIHYNYICNSSCMGGMNRR (SEQ ID NO: 94) (1 pg/mL) or WT p53-M237 peptide VGSDCTTIHYNYMCNSSCMGGMNRR (SEQ ID NO: 93) (1 pg/mL). Effector cells cultured alone and effector cells treated with PMA/ionomycin served as controls. Target cells treated with DMSO and transfected with all of the HLA Class II heterodimers shown in Table 11 served as a control. Target cells were also transfected with all of the HLA Class II heterodimers shown in Table 11 and pulsed with WT p53-M237 peptide as a control.

After co-culture of 20,000 effector cells with target cells for 18 hours, reactivity was tested by measuring IFN-gamma expression by ELISPOT assay. Reactivity was observed only upon co-culture of the 4304 TCR-4, 4304 TCR-K, or 4304 TCR-2-transduced cells with the p53-M237I 25-mer-loaded target cells which had been transduced with a nucleotide sequence encoding an HLA-DRA1*01:01/HLA-DRB1*01:01 heterodimer. These data show that 4304 TCR-4, 4304 TCR-K, and 4304 TCR-2 are restricted by the HLA-DRA1*01:01/HLA-DRB1*01:01 heterodimer.

### Example 14

This example demonstrates that the 4141 IVS TCR recognizes tumor cells in an HLA- and p53 mutation-specific manner.

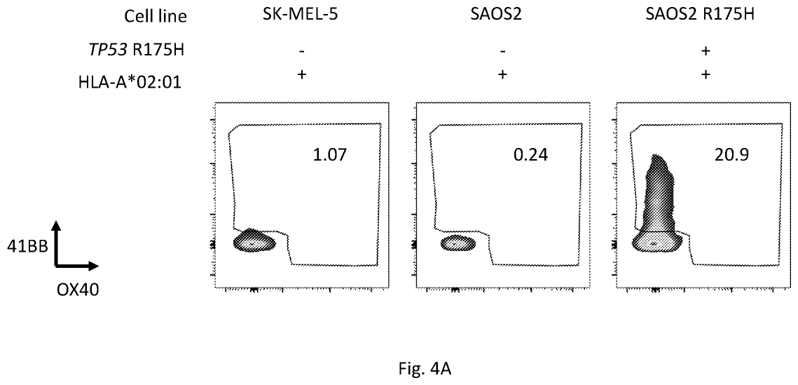

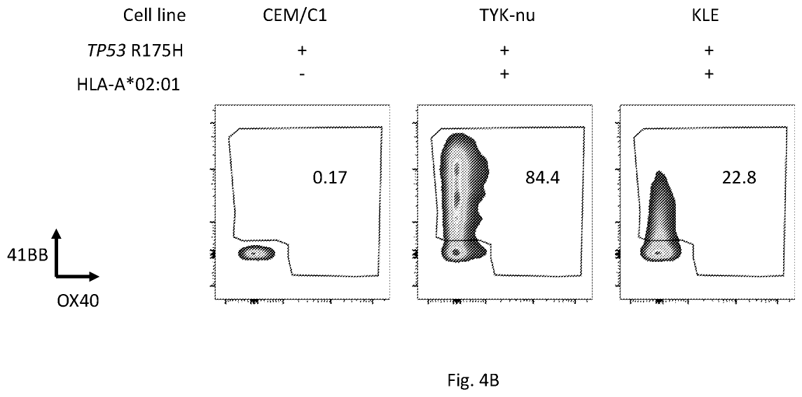

Healthy donor T cells transduced with the 4141 IVS TCR retroviral vector of Example 7 were co-cultured with a panel of tumor cell lines that were positive for p53 R175H or HLA-A*02:01 or both. T cell activation markers, 4-1BB and OX40, were measured in 4141 IVS TCR⁺ CD8⁺ T cells by flow cytometry. The results are shown in FIGS. 4A-4B. The results showed that the TCR-transduced cells recognized tumor cell lines that were positive for both p53 R175H and HLA-A*02:01. The TCR-transduced cells did not recognize the tumor cell lines that were negative for either of p53 R175H or HLA-A*02:01.

### Example 15

This example demonstrates autologous tumor cell recognition by the p53 C135Y-reactive 4316-D TCR.

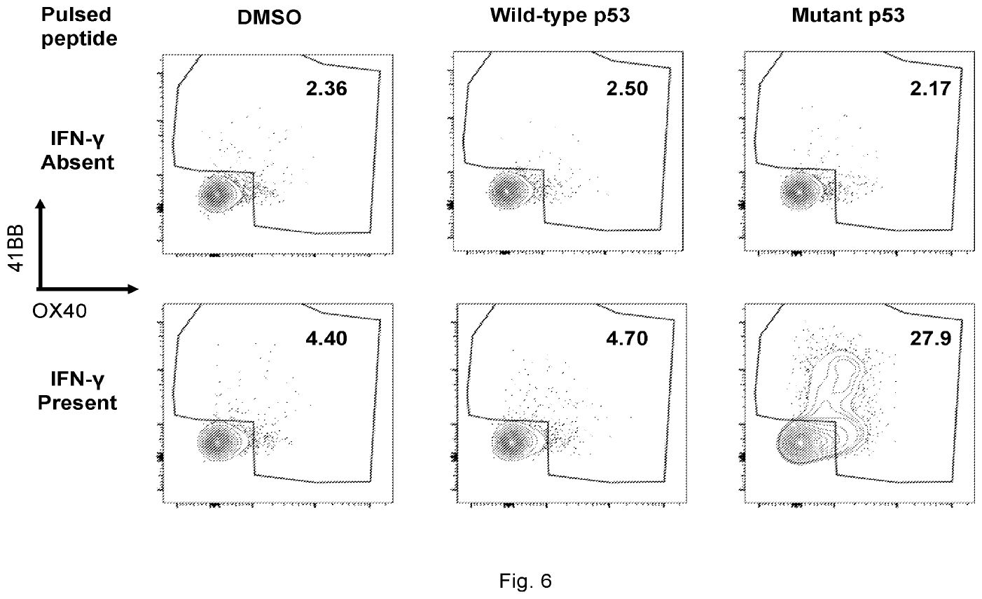

Healthy donor peripheral blood lymphocytes were retrovirally transduced with the 4316-D TCR retroviral vector of of Example 7. The ability of the transduced cells to recognize the autologous tumor cells was tested by co-culturing the transduced cells with target cells for 16 hours. The target cells were autologous PDX tumor cells from Patient 4316 pulsed with DMSO, the WT p53 of Example 8, or the mutant p53 peptide of Example 8 in the absence of IFN-γ or presence of IFN-γ. T cell activation was measured by flow cytometry using T cell activation markers, 4-1BB and OX40. The results are shown in FIG. 6. The results show that the transduced T cells upregulated 4-1BB and OX40 expression when co-cultured with the target cells that had been treated with both IFN-γ and the mutant p53 peptide.

### Example 16

This example demonstrates that 4141 IVS TCR exerts anti-tumor activity in a preclinical xenograft mouse model.

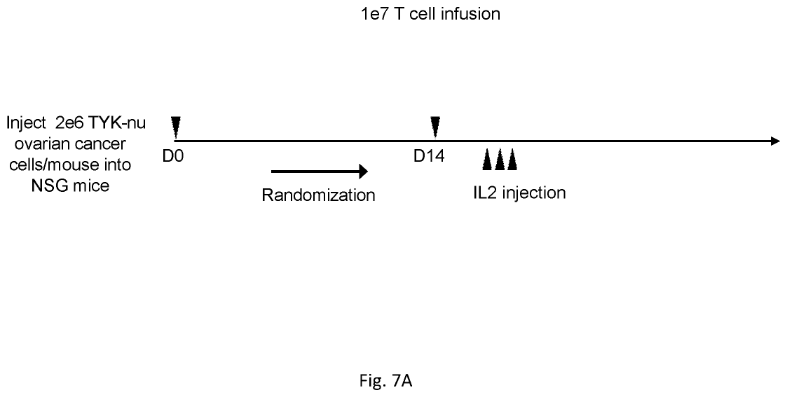

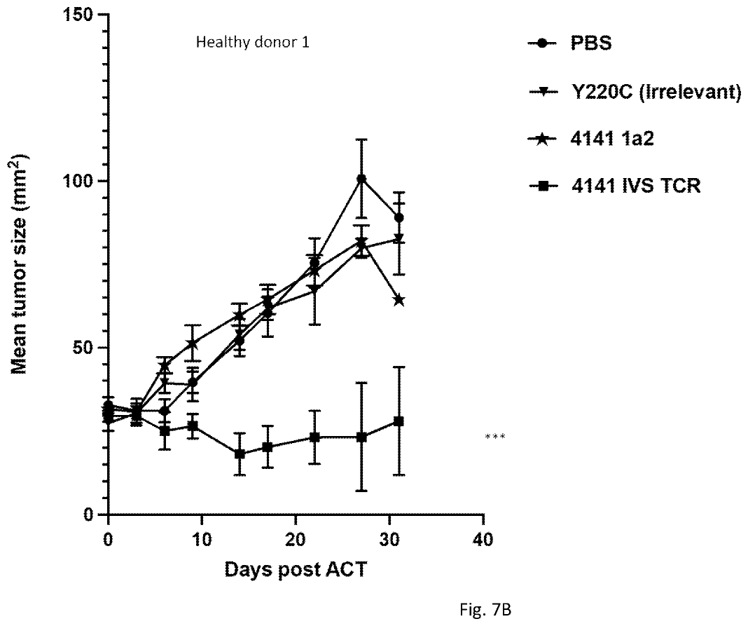

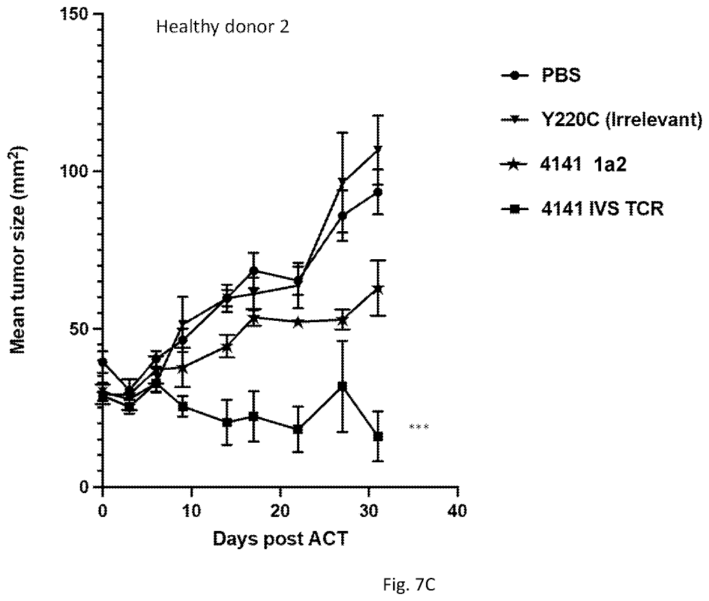

PBL from two healthy donors (healthy donor 1 and healthy donor 2) were independently transduced with the 4141 IVS TCR retroviral vector of Example 7. The anti-tumor activity of the transduced cells was compared to that of PBL transduced with the 4141-TCR1a2 (disclosed in U.S. patent application Ser. No. 17/620,942) using a preclinical mouse model. Female NSG mice were implanted with two million TYK-nu ovarian cancer cells that naturally expressed p53 R175H mutation and HLA-A*02:01. After 2 weeks, when the implanted tumor reached approximately 30 mm² in size, the mice were randomized and treated with a vehicle (PBS), ten million T cells transduced with an irrelevant TCR targeting p53 Y220C, or 10 million T cells transduced with 4141 IVS TCR (N=5). After the ACT treatment, these mice were given three daily I.V. injections of human recombinant interleukin 2 (IL-2) (180,000 IU). This process is shown in FIG. 7A. Over the 30 days following ACT, the mean tumor size of the mice was assessed and compared to the mean tumor size of mice treated with cells transduced with the 4141-TCR1a2. This experiment was performed twice with transduced PBL from two healthy donors (healthy donor 1 and healthy donor 2). The results of these experiments are shown in FIGS. 7B-7C. Mice treated with the 4141 IVS TCR-expressing T cells showed significantly delayed tumor growth relative to PBS or irrelevant TCR treated mice in both the healthy donor 1 and healthy donor 2 experiment. Treatment with the 4141 IVS TCR resulted in superior anti-tumor activity compared to treatment with the 4141-TCR1a2.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T cell receptor (TCR) having antigenic specificity for a human p53^{C135Y}, human p53^{R175H}, or human p53^{M237I} amino acid sequence, wherein the TCR comprises the amino acid sequences of:
(1) all of SEQ ID NOs: 2-4;
(2) all of SEQ ID NOs: 5-7;
(3) all of SEQ ID NOs: 2-7;
(4) all of SEQ ID NOs: 17-19;
(5) all of SEQ ID NOs: 20-22;
(6) all of SEQ ID NOs: 17-22;
(7) all of SEQ ID NOs: 32-34;
(8) all of SEQ ID NOs: 35-37;
(9) all of SEQ ID NOs: 32-37;
(10) all of SEQ ID NOs: 47-49;
(11) all of SEQ ID NOs: 50-52;
(12) all of SEQ ID NOs: 47-52;
(13) all of SEQ ID NOs: 62-64;
(14) all of SEQ ID NOs: 65-67; or
(15) all of SEQ ID NOs: 62-67.

2. The TCR of claim 1, wherein the TCR comprises the amino acid sequence(s) of:
(1) SEQ ID NO: 8;
(2) SEQ ID NO: 9;
(3) both of SEQ ID NOs: 8 and 9;
(4) SEQ ID NO: 10;
(5) SEQ ID NO: 11;
(6) both of SEQ ID NOs: 10 and 11;
(7) SEQ ID NO: 23;
(8) SEQ ID NO: 24;
(9) both of SEQ ID NOs: 23 and 24;
(10) SEQ ID NO: 25;
(11) SEQ ID NO: 26;
(12) both of SEQ ID NOs: 25 and 26;
(13) SEQ ID NO: 38;
(14) SEQ ID NOs: 39;
(15) both of SEQ ID NOs: 38 and 39;
(16) SEQ ID NO: 40;
(17) SEQ ID NO: 41;
(18) both of SEQ ID NOs: 40 and 41;
(19) SEQ ID NO: 53;
(20) SEQ ID NO: 54;
(21) both of SEQ ID NOs: 53 and 54;
(22) SEQ ID NO: 55;
(23) SEQ ID NO: 56;
(24) both of SEQ ID NOs: 55 and 56;
(25) SEQ ID NO: 68;
(26) SEQ ID NO: 69;
(27) both of SEQ ID NOs: 68 and 69;
(28) SEQ ID NO: 70;
(29) SEQ ID NO: 71; or
(30) both of SEQ ID NOs: 70 and 71.

3. The TCR of claim 1, wherein the TCR comprises the amino acid sequences of:
(1) SEQ ID NO: 12;
(2) SEQ ID NO: 13;
(3) both of SEQ ID NOs: 12 and 13;
(4) SEQ ID NO: 14;
(5) SEQ ID NO: 15;
(6) both of SEQ ID NOs: 14 and 15;
(7) SEQ ID NO: 27;
(8) SEQ ID NO: 28;
(9) both of SEQ ID NOs: 27 and 28;
(10) SEQ ID NO: 29;
(11) SEQ ID NO: 30;
(12) both of SEQ ID NOs: 29 and 30;
(13) SEQ ID NO: 42;
(14) SEQ ID NO: 43;
(15) both of SEQ ID NOs: 42 and 43;
(16) SEQ ID NO: 44;
(17) SEQ ID NO: 45;
(18) both of SEQ ID NOs: 44 and 45;
(19) SEQ ID NO: 57;
(20) SEQ ID NO: 58;
(21) both of SEQ ID NOs: 57 and 58;
(22) SEQ ID NO: 59;
(23) SEQ ID NO: 60;
(24) both of SEQ ID NOs: 59 and 60;
(25) SEQ ID NO: 72;
(26) SEQ ID NO: 73;
(27) both of SEQ ID NOs: 72 and 73;
(28) SEQ ID NO: 74;
(29) SEQ ID NO: 75; or
(30) both of SEQ ID NOs: 74 and 75.

4-9. (canceled)

10. An isolated or purified polypeptide comprising a functional portion of the TCR of claim 1, wherein the polypeptide comprises the amino acid sequences of:
(1) all of SEQ ID NOs: 2-4;
(2) all of SEQ ID NOs: 5-7;
(3) all of SEQ ID NOs: 2-7;
(4) all of SEQ ID NOs: 17-19;
(5) all of SEQ ID NOs: 20-22;
(6) all of SEQ ID NOs: 17-22;
(7) all of SEQ ID NOs: 32-34;
(8) all of SEQ ID NOs: 35-37;
(9) all of SEQ ID NOs: 32-37;
(10) all of SEQ ID NOs: 47-49;
(11) all of SEQ ID NOs: 50-52;
(12) all of SEQ ID NOs: 47-52;
(13) all of SEQ ID NOs: 62-64;
(14) all of SEQ ID NOs: 65-67; or
(15) all of SEQ ID NOs: 62-67.

11. The polypeptide of claim 10, wherein the polypeptide comprises the amino acid sequences of:
(1) SEQ ID NO: 8;
(2) SEQ ID NO: 9;
(3) both of SEQ ID NOs: 8 and 9;
(4) SEQ ID NO: 10;
(5) SEQ ID NO: 11;
(6) both of SEQ ID NOs: 10 and 11;
(7) SEQ ID NO: 23;
(8) SEQ ID NO: 24;
(9) both of SEQ ID NOs: 23 and 24;
(10) SEQ ID NO: 25;
(11) SEQ ID NO: 26;
(12) both of SEQ ID NOs: 25 and 26;
(13) SEQ ID NO: 38;
(14) SEQ ID NOs: 39;
(15) both of SEQ ID NOs: 38 and 39;
(16) SEQ ID NO: 40;
(17) SEQ ID NO: 41;
(18) both of SEQ ID NOs: 40 and 41;
(19) SEQ ID NO: 53;
(20) SEQ ID NO: 54;
(21) both of SEQ ID NOs: 53 and 54;
(22) SEQ ID NO: 55;
(23) SEQ ID NO: 56;
(24) both of SEQ ID NOs: 55 and 56;
(25) SEQ ID NO: 68;
(26) SEQ ID NO: 69;
(27) both of SEQ ID NOs: 68 and 69;
(28) SEQ ID NO: 70;
(29) SEQ ID NO: 71; or
(30) both of SEQ ID NOs: 70 and 71.

12. The polypeptide of claim 10, wherein the polypeptide comprises the amino acid sequences of:
(1) SEQ ID NO: 12;
(2) SEQ ID NO: 13;
(3) both of SEQ ID NOs: 12 and 13;
(4) SEQ ID NO: 14;
(5) SEQ ID NO: 15;
(6) both of SEQ ID NOs: 14 and 15;
(7) SEQ ID NO: 27;
(8) SEQ ID NO: 28;
(9) both of SEQ ID NOs: 27 and 28;
(10) SEQ ID NO: 29;
(11) SEQ ID NO: 30;
(12) both of SEQ ID NOs: 29 and 30;
(13) SEQ ID NO: 42;
(14) SEQ ID NO: 43;
(15) both of SEQ ID NOs: 42 and 43;
(16) SEQ ID NO: 44;
(17) SEQ ID NO: 45;
(18) both of SEQ ID NOs: 44 and 45;
(19) SEQ ID NO: 57;
(20) SEQ ID NO: 58;
(21) both of SEQ ID NOs: 57 and 58;
(22) SEQ ID NO: 59;
(23) SEQ ID NO: 60;
(24) both of SEQ ID NOs: 59 and 60;
(25) SEQ ID NO: 72;
(26) SEQ ID NO: 73;
(27) both of SEQ ID NOs: 72 and 73;
(28) SEQ ID NO: 74;
(29) SEQ ID NO: 75; or
(30) both of SEQ ID NOs: 74 and 75.

13. An isolated or purified protein comprising first and second polypeptide chains, wherein:
(1) the first polypeptide chain comprises the amino acid sequences of all of SEQ ID NOs: 2-4;
(2) the second polypeptide chain comprises the amino acid sequences of all of SEQ ID NOs: 5-7;
(3) the first polypeptide chain comprises the amino acid sequences of all of SEQ ID NOs: 2-4 and the second polypeptide chain comprises the amino acid sequences of all of SEQ ID NOs: 5-7;
(4) the first polypeptide chain comprises the amino acid sequences of all of SEQ ID NOs: 17-19;
(5) the second polypeptide chain comprises the amino acid sequences of all of SEQ ID NOs: 20-22;
(6) the first polypeptide chain comprises the amino acid sequences of all of SEQ ID NOs: 17-19 and the second polypeptide chain comprises the amino acid sequences of all of SEQ ID NOs: 20-22;
(7) the first polypeptide chain comprises the amino acid sequences of all of SEQ ID NOs: 32-34;
(8) the second polypeptide chain comprises the amino acid sequences of all of SEQ ID NOs: 35-37;
(9) the first polypeptide chain comprises the amino acid sequences of all of SEQ ID NOs: 32-34 and the second polypeptide chain comprises the amino acid sequences of all of SEQ ID NOs: 35-37;
(10) the first polypeptide chain comprises the amino acid sequences of all of SEQ ID NOs: 47-49;
(11) the second polypeptide chain comprises the amino acid sequences of all of SEQ ID NOs: 50-52;
(12) the first polypeptide chain comprises the amino acid sequences of all of SEQ ID NOs: 47-49 and the second polypeptide chain comprises the amino acid sequences of all of SEQ ID NOs: 50-52;
(13) the first polypeptide chain comprises the amino acid sequences of all of SEQ ID NOs: 62-64;
(14) the second polypeptide chain comprises the amino acid sequences of all of SEQ ID NOs: 65-67; or
(15) the first polypeptide chain comprises the amino acid sequences of all of SEQ ID NOs: 62-64 and the second polypeptide chain comprises the amino acid sequences of all of SEQ ID NOs: 65-67.

14. The protein of claim 13, wherein:
(1) the first polypeptide chain comprises the amino acid sequence of SEQ ID NO: 8;
(2) the second polypeptide chain comprises the amino acid sequence of SEQ ID NO: 9;
(3) the first polypeptide chain comprises the amino acid sequence of SEQ ID NO: 8 and the second polypeptide chain comprises the amino acid sequence of SEQ ID NO: 9;
(4) the first polypeptide chain comprises the amino acid sequence of SEQ ID NO: 10;
(5) the second polypeptide chain comprises the amino acid sequence of SEQ ID NO: 11;
(6) the first polypeptide chain comprises the amino acid sequence of SEQ ID NO: 10 and the second polypeptide chain comprises the amino acid sequence of SEQ ID NO: 11;
(7) the first polypeptide chain comprises the amino acid sequence of SEQ ID NO: 23;
(8) the second polypeptide chain comprises the amino acid sequence of SEQ ID NO: 24;
(9) the first polypeptide chain comprises the amino acid sequence of SEQ ID NO: 23 and the second polypeptide chain comprises the amino acid sequence of SEQ ID NO: 24;
(10) the first polypeptide chain comprises the amino acid sequence of SEQ ID NO: 25;
(11) the second polypeptide chain comprises the amino acid sequence of SEQ ID NO: 26;
(12) the first polypeptide chain comprises the amino acid sequence of SEQ ID NO: 25 and the second polypeptide chain comprises the amino acid sequence of SEQ ID NO: 26;
(13) the first polypeptide chain comprises the amino acid sequence of SEQ ID NO: 38;
(14) the second polypeptide chain comprises the amino acid sequence of SEQ ID NO: 39;
(15) the first polypeptide chain comprises the amino acid sequence of SEQ ID NO: 38 and the second polypeptide chain comprises the amino acid sequence of SEQ ID NO: 39;
(16) the first polypeptide chain comprises the amino acid sequence of SEQ ID NO: 40;
(17) the second polypeptide chain comprises the amino acid sequence of SEQ ID NO: 41;
(18) the first polypeptide chain comprises the amino acid sequence of SEQ ID NO: 40 and the second polypeptide chain comprises the amino acid sequence of SEQ ID NO: 41;
(19) the first polypeptide chain comprises the amino acid sequence of SEQ ID NO: 53;
(20) the second polypeptide chain comprises the amino acid sequence of SEQ ID NO: 54;
(21) the first polypeptide chain comprises the amino acid sequence of SEQ ID NO: 53 and the second polypeptide chain comprises the amino acid sequence of SEQ ID NO: 54;
(22) the first polypeptide chain comprises the amino acid sequence of SEQ ID NO: 55;
(23) the second polypeptide chain comprises the amino acid sequence of SEQ ID NO: 56;
(24) the first polypeptide chain comprises the amino acid sequence of SEQ ID NO: 55 and the second polypeptide chain comprises the amino acid sequence of SEQ ID NO: 56;
(25) the first polypeptide chain comprises the amino acid sequence of SEQ ID NO: 68;
(26) the second polypeptide chain comprises the amino acid sequence of SEQ ID NO: 69;
(27) the first polypeptide chain comprises the amino acid sequence of SEQ ID NO: 68 and the second polypeptide chain comprises the amino acid sequence of SEQ ID NO: 69;
(28) the first polypeptide chain comprises the amino acid sequence of SEQ ID NO: 70;
(29) the second polypeptide chain comprises the amino acid sequence of SEQ ID NO: 71; or
(30) the first polypeptide chain comprises the amino acid sequence of SEQ ID NO: 70 and the second polypeptide chain comprises the amino acid sequence of SEQ ID NO: 71.

15. The protein of claim 13, wherein:
(1) the first polypeptide chain comprises the amino acid sequence of SEQ ID NO: 12;
(2) the second polypeptide chain comprises the amino acid sequence of SEQ ID NO: 13;
(3) the first polypeptide chain comprises the amino acid sequence of SEQ ID NO: 12 and the second polypeptide chain comprises the amino acid sequence of SEQ ID NO: 13;
(4) the first polypeptide chain comprises the amino acid sequence of SEQ ID NO: 14;
(5) the second polypeptide chain comprises the amino acid sequence of SEQ ID NO: 15;
(6) the first polypeptide chain comprises the amino acid sequence of SEQ ID NO: 14 and the second polypeptide chain comprises the amino acid sequence of SEQ ID NO: 15;
(7) the first polypeptide chain comprises the amino acid sequence of SEQ ID NO: 27;
(8) the second polypeptide chain comprises the amino acid sequence of SEQ ID NO: 28;
(9) the first polypeptide chain comprises the amino acid sequence of SEQ ID NO: 27 and the second polypeptide chain comprises the amino acid sequence of SEQ ID NO: 28;
(10) the first polypeptide chain comprises the amino acid sequence of SEQ ID NO: 29;
(11) the second polypeptide chain comprises the amino acid sequence of SEQ ID NO: 30;
(12) the first polypeptide chain comprises the amino acid sequence of SEQ ID NO: 29 and the second polypeptide chain comprises the amino acid sequence of SEQ ID NO: 30;
(13) the first polypeptide chain comprises the amino acid sequence of SEQ ID NO: 42;
(14) the second polypeptide chain comprises the amino acid sequence of SEQ ID NO: 43;
(15) the first polypeptide chain comprises the amino acid sequence of SEQ ID NO: 42 and the second polypeptide chain comprises the amino acid sequence of SEQ ID NO: 43;
(16) the first polypeptide chain comprises the amino acid sequence of SEQ ID NO: 44;
(17) the second polypeptide chain comprises the amino acid sequence of SEQ ID NO: 45;
(18) the first polypeptide chain comprises the amino acid sequence of SEQ ID NO: 44 and the second polypeptide chain comprises the amino acid sequence of SEQ ID NO: 45;
(19) the first polypeptide chain comprises the amino acid sequence of SEQ ID NO: 57;
(20) the second polypeptide chain comprises the amino acid sequence of SEQ ID NO: 58;
(21) the first polypeptide chain comprises the amino acid sequence of SEQ ID NO: 57 and the second polypeptide chain comprises the amino acid sequence of SEQ ID NO: 58;
(22) the first polypeptide chain comprises the amino acid sequence of SEQ ID NO: 59;
(23) the second polypeptide chain comprises the amino acid sequence of SEQ ID NO: 60;
(24) the first polypeptide chain comprises the amino acid sequence of SEQ ID NO: 59 and the second polypeptide chain comprises the amino acid sequence of SEQ ID NO: 60;
(25) the first polypeptide chain comprises the amino acid sequence of SEQ ID NO: 72;
(26) the second polypeptide chain comprises the amino acid sequence of SEQ ID NO: 73;
(27) the first polypeptide chain comprises the amino acid sequence of SEQ ID NO: 72 and the second polypeptide chain comprises the amino acid sequence of SEQ ID NO: 73;
(28) the first polypeptide chain comprises the amino acid sequence of SEQ ID NO: 74;
(29) the second polypeptide chain comprises the amino acid sequence of SEQ ID NO: 75; or
(30) the first polypeptide chain comprises the amino acid sequence of SEQ ID NO: 74 and the second polypeptide chain comprises the amino acid sequence of SEQ ID NO: 75.

16. An isolated or purified nucleic acid comprising a nucleotide sequence encoding the TCR of claim 1.

17. An isolated or purified nucleic acid comprising, from 5′ to 3′, a first nucleic acid sequence and a second nucleotide sequence, wherein the first and second nucleotide sequence, respectively, encode the amino sequences of SEQ ID NOs: 8 and 9; 9 and 8; 10 and 11; 11 and 10; 12 and 13; 13 and 12; 14 and 15; 15 and 14; 23 and 24; 24 and 23; 25 and 26; 26 and 25; 27 and 28; 28 and 27; 29 and 30; 30 and 29; 38 and 39; 39 and 38; 40 and 41; 41 and 40; 42 and 43; 43 and 42; 44 and 45; 45 and 44; 53 and 54; 54 and 53; 55 and 56; 56 and 55; 57 and 58; 58 and 57; 59 and 60; 60 and 59; 68 and 69; 69 and 68; 70 and 71; 71 and 70; 72 and 73; 73 and 72; 74 and 75; or 75 and 74.

18-20. (canceled)

21. A recombinant expression vector comprising the nucleic acid of claim 16.

22-24. (canceled)

25. A method of producing a host cell expressing a TCR that has antigenic specificity for a human p53^{C135Y}, human p53^{R175H}, or human p53^{M237I} amino acid sequence, the method comprising contacting a cell in vitro with the vector of claim 21 under conditions that allow introduction of the vector into the cell.

26. An isolated or purified host cell comprising the nucleic acid of claim 16.

27-28. (canceled)

29. An isolated or purified population of cells comprising the host cell of claim 26.

30. A method of producing a TCR, the method comprising culturing the host cell of claim 26 so that the TCR is produced.

31. A pharmaceutical composition comprising (a) the population of host cells of claim 29 and (b) a pharmaceutically acceptable carrier.

32. A method of detecting the presence of cancer in a mammal, the method comprising:
(a) contacting a sample comprising cells of the cancer with the TCR claim 1, thereby forming a complex; and
(b) detecting the complex,
wherein detection of the complex is indicative of the presence of cancer in the mammal.

33-39. (canceled)

40. A method of inducing an immune response against a cancer in a mammal, comprising administering to the mammal the population of host cells of claim 29 in an amount effective to induce an immune response against the cancer in the mammal.

41. A method of treating or preventing cancer in a mammal, comprising administering to the mammal the population of host cells of claim 29 in an amount effective to treat or prevent cancer in the mammal.
